SEQUENCE LISTING (31 sequences)

SEQ ID NO: 1 - RNA, 22 nt, synthetic construct
  source 1..22: mol_type = other RNA
aatgcacccgggcaaggattct

SEQ ID NO: 2 - RNA, 22 nt, synthetic construct
  source 1..22: mol_type = other RNA
aatgcacctgggcaaggattca

SEQ ID NO: 3 - RNA, 20 nt, synthetic construct
  source 1..20: mol_type = other RNA
gtagaggagatggcgcaggg

SEQ ID NO: 4 - RNA, 22 nt, synthetic construct
  source 1..22: mol_type = other RNA
atgcacctgggcaaggattctg

SEQ ID NO: 5 - RNA, 22 nt, synthetic construct
  source 1..22: mol_type = other RNA
ttcatttgcctcccagcctaca

SEQ ID NO: 6 - RNA, 17 nt, synthetic construct
  source 1..17: mol_type = other RNA
gcggggctgggcgcgcg

SEQ ID NO: 7 - RNA, 21 nt, synthetic construct
  source 1..21: mol_type = other RNA
cgggggcggggccgaagcgcg

SEQ ID NO: 8 - RNA, 24 nt, synthetic construct
  source 1..24: mol_type = other RNA
agggctggactcagcggcggagct

SEQ ID NO: 9 - RNA, 17 nt, synthetic construct
  source 1..17: mol_type = other RNA
ctccgggacggctgggc

SEQ ID NO: 10 - RNA, 23 nt, synthetic construct
  source 1..23: mol_type = other RNA
agcagcattgtacagggctatga

SEQ ID NO: 11 - RNA, 23 nt, synthetic construct
  source 1..23: mol_type = other RNA
agcagcattgtacagggctatga

SEQ ID NO: 12 - RNA, 22 nt, synthetic construct
  source 1..22: mol_type = other RNA
taaggcacgcggtgaatgccaa

SEQ ID NO: 13 - RNA, 22 nt, synthetic construct
  source 1..22: mol_type = other RNA
tggctcagttcagcaggaacag

SEQ ID NO: 14 - RNA, 22 nt, synthetic construct
  source 1..22: mol_type = other RNA
tgaggtagtaggttgtatagtt

SEQ ID NO: 15 - RNA, 22 nt, synthetic construct
  source 1..22: mol_type = other RNA
tggagagaaaggcagttcctga

SEQ ID NO: 16 - RNA, 22 nt, synthetic construct
  source 1..22: mol_type = other RNA
aaaagctgggttgagagggcaa

SEQ ID NO: 17 - RNA, 22 nt, synthetic construct
  source 1..22: mol_type = other RNA
agaggtagtaggttgcatagtt

SEQ ID NO: 18 - RNA, 22 nt, synthetic construct
  source 1..22: mol_type = other RNA
taccacagggtagaaccacgga

SEQ ID NO: 19 - RNA, 23 nt, synthetic construct
  source 1..23: mol_type = other RNA
caaagtgcttacagtgcaggtag

SEQ ID NO: 20 - RNA, 21 nt, synthetic construct
  source 1..21: mol_type = other RNA
tcgaggagctcacagtctagt

SEQ ID NO: 21 - RNA, 18 nt, synthetic construct
  source 1..18: mol_type = other RNA
cggggcggcaggggcctc

SEQ ID NO: 22 - RNA, 24 nt, synthetic construct
  source 1..24: mol_type = other RNA
ctgcaggcagaagtggggctgaca

SEQ ID NO: 23 - RNA, 17 nt, synthetic construct
  source 1..17: mol_type = other RNA
gcggggctgggcgcgcg

SEQ ID NO: 24 - RNA, 21 nt, synthetic construct
  source 1..21: mol_type = other RNA
tccttctgctccgtcccccag

SEQ ID NO: 25 - RNA, 24 nt, synthetic construct
  source 1..24: mol_type = other RNA
agggctggactcagcggcggagct

SEQ ID NO: 26 - RNA, 17 nt, synthetic construct
  source 1..17: mol_type = other RNA
ggggctgggcgcgcgcc

SEQ ID NO: 27 - RNA, 17 nt, synthetic construct
  source 1..17: mol_type = other RNA
aagactgagaggaggga

SEQ ID NO: 28 - RNA, 17 nt, synthetic construct
  source 1..17: mol_type = other RNA
ctccgggacggctgggc

SEQ ID NO: 29 - RNA, 23 nt, synthetic construct
  source 1..23: mol_type = other RNA
agcagcattgtacagggctatca

SEQ ID NO: 30 - RNA, 23 nt, synthetic construct
  source 1..23: mol_type = other RNA
agctggtgttgtgaatcaggccg

SEQ ID NO: 31 - RNA, 22 nt, synthetic construct
  source 1..22: mol_type = other RNA
ttacgtggacccgttcctaagt

(19) United States
(12) Patent Application Publication
(10) Pub. No.: US 2025/0270565 A1
(43) Pub. Date: Aug. 28, 2025

(54) SYNAPTOSOMAL MICRO RNAS AND SYNAPSE FUNCTIONS IN ALZHEIMER'S DISEASE

(71) Applicant: Texas Tech University System, Lubbock, TX (US)

(72) Inventors: Subodh Kumar, Lubbock, TX (US); P. Hemachandra Reddy, Lubbock, TX (US)

(73) Assignee: Texas Tech University System, Lubbock, TX (US)

(21) Appl. No.: 18/858,310

(22) PCT Filed: Apr. 20, 2023
(86) PCT No.: PCT/US2023/019298

§ 371 Date: Oct. 19, 2024

Related U.S. Application Data
(60) Provisional application No. 63/332,866, filed on Apr. 20, 2022.

(51) Int. Cl. C12N 15/113; A61P 25/28

(52) U.S. Cl. CPC C12N 15/1138; A61P 25/28; C12N 2310/141; C12N 2320/30

(57) ABSTRACT

Embodiments of the present disclosure pertain to methods of treating or preventing a neurological disorder in a subject by administering to the subject at least one microRNA (miR-NA), at least one inhibitor of the miRNA, or combinations thereof. Additional embodiments of the present disclosure pertain to compositions that include at least one miRNA of the present disclosure, at least one inhibitor of the miRNAs of the present disclosure, or combinations thereof. In some embodiments, the composition is suitable for use in treating or preventing a neurological disorder in a subject.

### CROSS-REFERENCE TO RELATED APPLICATIONS

This application claims priority to U.S. Provisional Patent Application No. 63/332,866, filed on Apr. 20, 2022. The entirety of the aforementioned application is incorporated herein by reference.

### STATEMENT UNDER 37 C.F.R. § 1.834(C)(1)

Pursuant to 37 C.F.R. § 1.834, Applicant hereby submits a sequence listing as an XML file (“Sequence Listing”). The name of the file containing the Sequence Listing is “AF13368.P037WO.xml”. The date of the creation of the Sequence Listing is Apr. 20, 2023. The size of the Sequence Listing is 28,000 bytes. Applicant hereby incorporates by reference the material in the Sequence Listing.

### BACKGROUND

Current methods and therapeutics for treating or preventing neurodegenerative diseases have numerous limitations. Embodiments of the present disclosure aim to address the aforementioned limitations.

### SUMMARY

Embodiments of the present disclosure pertain to methods of treating or preventing a neurological disorder in a subject by administering to the subject at least one microRNA (miRNA), at least one inhibitor of the miRNA, or combinations thereof. In some embodiments, the miRNA to be administered or inhibited includes, without limitation, miR-501-3p (SEQ ID NO: 1), miR-502-3p (SEQ ID NO: 2), miR-877-5p (SEQ ID NO: 3), miR-500a-3p (SEQ ID NO: 4), miR-664b-3p (SEQ ID NO: 5), miR-4508 (SEQ ID NO: 6), miR-1237-5p (SEQ ID NO: 7), miR-5001-5p (SEQ ID NO: 8), miR-4497 (SEQ ID NO: 9), miR-103-3p (SEQ ID NO: 10), miR-103a-3p (SEQ ID NO: 11), miR-124-3p (SEQ ID NO: 12), miR-24-3p (SEQ ID NO: 13), let-7a-5p (SEQ ID NO: 14), miR-185-5p (SEQ ID NO: 15), miR-320b (SEQ ID NO: 16), let-7d-5p (SEQ ID NO: 17), miR-140-3p (SEQ ID NO: 18), miR-17-5p (SEQ ID NO: 19), miR-151a-5p (SEQ ID NO: 20), miR-3196 (SEQ ID NO: 21), miR-651 lb-5p (SEQ ID NO: 22), miR-4508 (SEQ ID NO: 23), miR-1237-3p (SEQ ID NO: 24), miR-5001-5p (SEQ ID NO: 25), miR-4492 (SEQ ID NO: 26), miR-4499 (SEQ ID NO: 27), miR-4497 (SEQ ID NO: 28), miR-107 (SEQ ID NO: 29), miR-138-5p (SEQ ID NO: 30), derivatives thereof, or combinations thereof.

The methods of the present disclosure may be utilized to treat or prevent various neurological disorders. For instance, in some embodiments, the neurological disorder includes, without limitation, Alzheimer's Disease (AD), Huntington's Disease, Parkinson's Disease, Amyotrophic Lateral Sclerosis (ALS), Multiple Sclerosis (MS), dementia, mild cognitive impairment (MCI), Schizophrenia, or combinations thereof. In some embodiments, the neurological disorder to be treated or prevented includes Alzheimer's Disease (AD).

Additional embodiments of the present disclosure pertain to compositions that include at least one miRNA of the present disclosure, at least one inhibitor of the miRNAs of the present disclosure, or combinations thereof. In some embodiments, the composition is suitable for use in treating or preventing a neurological disorder in a subject.

### DESCRIPTION OF THE DRAWINGS

FIGS. 1A-1G illustrate the extraction and characterization of Synaptosomes.

FIG. 1A provides a brief workflow of the extraction and characterization process.

FIG. 1B shows immunoblotting analysis of synaptic (SNAP25, Synaptophysin and PSD95) and cytosolic (elF1a and PCNA) proteins in cytosolic fraction, synaptosomal fraction and leftover tissue debris of unaffected control postmortem brain tissues (n=2).

FIG. 1C shows densitometry analysis of synaptic and cytosolic proteins. Synaptic proteins levels (PSD95; p=0.003), (SNAP25; p=0.0061), (Synaptophysin; p=0.026) were significantly higher in synaptosomes and cytosolic proteins (elF1a; p=0.012) and (PCNA; p=0.018) levels were significantly lower in synaptosomes relative to cytosol.

FIG. 1D shows qRT-PCR analysis for mRNA fold change analysis of synaptic and cytosolic genes in cytosolic and synaptosomal fractions (n=5).

FIG. 1E shows TEM analysis of synapse assembly in synaptosomal fraction from unaffected control and AD patients postmortem brains (500 and 100 nm magnification). Arrows indicate the synapse components (Mt—mitochondria, Sv—synaptic vesicles, PSD—postsynaptic density, and Sc—synaptic cleft).

FIG. 1F shows immunoblotting analysis of brain cells markers (Neuron-NeuN; Microglia-Iba1), excitatory synapse marker (VGLUT1) and inhibitory synapse marker (GABARA1) proteins in unaffected controls (n=4) and AD (n=4) synaptosomes.

FIG. 1G shows densitometry analysis of NeuN, Iba1, VGLUT1 and GABARA1 proteins in unaffected controls and AD synaptosomes.

FIGS. 2A-2E show miRNA expression in synaptosome and cytosol in healthy states.

FIG. 2A shows a hierarchical clustering and heat map of significantly deregulated miRNAs in the synaptosome and cytosol of unaffected controls. Dark color intensity showed miRNAs upregulation and light color intensity showed miRNAs downregulation.

FIG. 2B shows the total number of miRNAs deregulated in cytosol vs synaptosome in unaffected controls.

FIG. 2C provides a Pi diagram showing the total miRNAs pool distribution and percentage of miRNAs population changed in cytosol and synaptosome in unaffected controls.

FIG. 2D shows qRT-PCR based validation analysis of significantly deregulated miRNAs in unaffected controls (n=15). MiRNAs expression was quantified in terms of fold changes in unaffected controls synaptosomes compared to cytosol. Each circle dot represents one sample.

FIG. 2E shows validation analysis of significantly deregulated mmu-miRNAs in WT mice (n=7). MiRNAs expression was quantified in synaptosome relative to cytosol. Each circle dot represents one animal.

FIGS. 3A-3F show miRNAs expression in synaptosome and cytosol in Alzheimer's Disease (AD).

FIG. 3A shows hierarchical clustering and heat map of significantly deregulated miRNAs in cytosol and synaptosome in AD samples. Dark color intensity showed miRNAs upregulation and light color intensity showed the miRNAs downregulation.

FIG. 3B shows total numbers of miRNAs deregulated in cytosol and synaptosome in AD.

FIG. 3C provides a Pi diagram showing the total miRNAs pool distribution and percentage of miRNA populations changed in cytosol and synaptosome.

FIG. 3D shows qRT-PCR based validation analysis of significantly deregulated miRNAs in AD samples (n=27). MiRNAs expression was quantified in terms of fold changes in AD synaptosome compared to AD cytosol. Each circle dot represents one sample.

FIG. 3E shows validation analysis of significantly deregulated mmu-miRNAs in APP Tg (n=6) mice. MiRNAs expression was quantified in synaptosome relative to cytosol. Each circle dot represents one animal.

FIG. 3F shows validation analysis of significantly deregulated mmu-miRNAs in Tau Tg (n=7) mice. MiRNAs expression was quantified in synaptosome relative to cytosol.

FIGS. 4A-4H show miRNAs expression in synaptosomes in AD and healthy states.

FIG. 4A shows hierarchical clustering and heat map of significantly deregulated miRNAs in synaptosome in AD and unaffected controls. Dark color intensity showed miRNAs upregulation and light color intensity showed miRNAs downregulation.

FIG. 4B shows the total numbers of miRNAs deregulated in AD synaptosome vs UC synaptosome.

FIG. 4C provides a Pi diagram showing the total miRNAs pool distribution and percentage of miRNAs population changed in AD synaptosome vs UC synaptosome.

FIG. 4D shows a Venn diagram showing the number of miRNAs that expressed only in cytosol and synaptosome in AD vs healthy state.

FIG. 4E shows a qRT-PCR based validation analysis of significantly deregulated miRNAs in AD (n=27) and UC (n=15) synaptosome. MiRNAs expression was quantified in terms of fold changes in AD synaptosome relative to UC synaptosome. Each circle dot represents one sample.

FIG. 4F shows multiple comparison analysis of synaptosomal miRNAs fold changes with Braak stages 2/3, Braak stages 4/5 and Braak stages 6 of AD samples. (**p<0.01, ***p<0.001, ****p<0.0001).

FIG. 4G shows immunoblotting analysis of miRNAs biogenesis proteins (Ago2, Drosha and Dicer) in cytosol and synaptosomal of AD samples (n=4).

FIG. 4H shows densitometry analysis of Ago2, Drosha and Dicer in cytosol relative to synaptosomes of UC samples.

FIGS. 5A-5D show an Ingenuity Pathway Analysis of cytosolic and synaptosome miRNAs in AD and healthy state.

FIG. 5A shows the healthy state analysis of the cytosolic and synaptosome miRNAs expression network in various human diseases. Light nodes represent decreased expression and dark nodes represent increased expression of miRNAs.

FIG. 5B shows miRNAs target and seed sequences network of cytosolic and synaptosome miRNAs in healthy state.

FIG. 5C shows the AD state analysis of cytosolic and synaptosome miRNAs expression network in various human diseases. Light nodes represent decreased expression and dark nodes represent increased expression of miRNAs.

FIG. 5D shows miRNAs target and seed sequences network of cytosolic and synaptosome miRNAs in AD state.

FIGS. 6A-6C shows Ingenuity Pathway Analysis of synaptosomal miRNAs in AD.

FIG. 6A shows synaptosomal miRNAs expression network in various human diseases.

FIG. 6B shows miRNAs target and seed sequences network of synaptosomal miRNAs in AD and healthy state. Light nodes represent increased expression and dark nodes represent decreased expression of miRNAs.

FIG. 6C shows possible molecular mechanism of GABAergic synapse regulation by miR-501-3p, miR-502-3p and miR-877-5p. KRAS gene is one of the top predicted targets of miR-501-3p, miR-502-3p and miR-877-5p. Inhibition of KRAS expression by the overexpression of these miRNAs could inhibit the GABAregic synapse function in AD.

FIGS. 7A1-7C3 summarizes experimental results related to the impact of miR-502-3p on cell viability and cell survival of mouse primary hippocampal neurons.

FIGS. 8A-8B summarize experimental results related to the impact of miR-502-3p on GABAergic receptor 1 alpha (GABARA1) expression.

FIGS. 9A-9B summarize experimental results related to the impact of miR-502-3p on GABAergic receptor 1 alpha (GABARA1) protein levels.

FIGS. 10A-10C summarize experimental results related to the impact of miR-502-3p on GABAergic receptor 1 alpha (GABARA1) protein levels.

FIGS. 11A-11D summarize experimental results related to the impact of miR-502-3p on GABAergic receptor 1 alpha (GABARA1) protein levels.

FIGS. 12A-12E provide electrophysiology-patch clamp analyses of m-cells transfected with miR-502-3p agomiRs (mimics) and antagomiRs (inhibitors).

FIGS. 13A-13F show light microscopy images of mouse hippocampal neurons (HT22 cells).

FIG. 14 summarizes cell viability analyses of HT22 cells transfected with GABRA1 expression vector, miR-502-3p overexpression vector, and miR-502-3p sponge suppression plasmids.

FIGS. 15A-15C summarize QRT-PCR analysis of miR-502-3p, GABRA1 and VGLUT1 in cells transfected with GABRA1 expression vector, miR-502-3p overexpression vector and miR-502-3p sponge suppression plasmids.

FIGS. 16A-16B summarize immunoblot (FIG. 16A) and quantitative (FIG. 168) analyses of GABRA1 in HT22 cells transfected with GABRA1 overexpression vector, miR-502-3p overexpression vector and miR-502-3p sponge suppression plasmids.

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that include more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

Neurodegenerative diseases present a public health crisis. For instance, Alzheimer's disease (AD) progresses with synaptic failure caused by amyloid beta (AR) and phosphorylated tau (p-tau) toxicities at synapses. In aged individuals, the numbers of AD cases are increasing gradually, and by mid-century, the number of Americans aged 65 and older with Alzheimer's dementia may grow to 13.8 million. This represents a steep increase from the estimated 5.8 million Americans aged 65 and older who have Alzheimer's dementia today.

However, current methods and therapeutics for treating or preventing neurodegenerative diseases have numerous limitations. Embodiments of the present disclosure aim to address the aforementioned limitations.

### Methods of Treating or Preventing Neurological Disorders

In some embodiments, the present disclosure pertains to methods of treating or preventing a neurological disorder in a subject. In some embodiments, the methods of the present disclosure include administering to the subject at least one microRNA (miRNA), at least one inhibitor of the miRNA, or combinations thereof. In some embodiments, the miRNA to be administered or inhibited includes, without limitation, miR-501-3p (SEQ ID NO: 1), miR-502-3p (SEQ ID NO: 2), miR-877-5p (SEQ ID NO: 3), miR-500a-3p (SEQ ID NO: 4), miR-664b-3p (SEQ ID NO: 5), miR-4508 (SEQ ID NO: 6), miR-1237-5p (SEQ ID NO: 7), miR-5001-5p (SEQ ID NO: 8), miR-4497 (SEQ ID NO: 9), miR-103-3p (SEQ ID NO: 10), miR-103a-3p (SEQ ID NO: 11), miR-124-3p (SEQ ID NO: 12), miR-24-3p (SEQ ID NO: 13), let-7a-5p (SEQ ID NO: 14), miR-185-5p (SEQ ID NO: 15), miR-320b (SEQ ID NO: 16), let-7d-5p (SEQ ID NO: 17), miR-140-3p (SEQ ID NO: 18), miR-17-5p (SEQ ID NO: 19), miR-151a-5p (SEQ ID NO: 20), miR-3196 (SEQ ID NO: 21), miR-6511b-5p (SEQ ID NO: 22), miR-4508 (SEQ ID NO: 23), miR-1237-3p (SEQ ID NO: 24), miR-5001-5p (SEQ ID NO: 25), miR-4492 (SEQ ID NO: 26), miR-4499 (SEQ ID NO: 27), miR-4497 (SEQ ID NO: 28), miR-107 (SEQ ID NO: 29), miR-138-5p (SEQ ID NO: 30), derivatives thereof, or combinations thereof.

In some embodiments, the miRNA to be administered or inhibited includes a derivative of any one of the aforementioned miRNAs. In some embodiments, the miRNA derivative shares at least 70% sequence identity with any of the aforementioned miRNAs. In some embodiments, the miRNA derivative shares at least 75% sequence identity with any of the aforementioned miRNAs. In some embodiments, the miRNA derivative shares at least 80% sequence identity with any of the aforementioned miRNAs. In some embodiments, the miRNA derivative shares at least 85% sequence identity with any of the aforementioned miRNAs. In some embodiments, the miRNA derivative shares at least 90% sequence identity with any of the aforementioned miRNAs. In some embodiments, the miRNA derivative shares at least 95% sequence identity with any of the aforementioned miRNAs. In some embodiments, the miRNA derivative shares at least 99% sequence identity with any of the aforementioned miRNAs.

In some embodiments, the methods of the present disclosure include a step of administering at least one miRNA. In some embodiments, the administered miRNA includes, without limitation, miR-3196 (SEQ ID NO: 21), miR-651 lb-5p (SEQ ID NO: 22), miR-4508 (SEQ ID NO: 23), miR-1237-5p (SEQ ID NO: 7), miR-5001-5p (SEQ ID NO: 8), miR-4492 (SEQ ID NO: 26), miR-4499 (SEQ ID NO: 27), miR-4497 (SEQ ID NO: 28), miR 107 (SEQ ID NO: 29), derivatives thereof, or combinations thereof.

In some embodiments, the methods of the present disclosure include a step of administering at least one inhibitor of a miRNA. In some embodiments, the administered miRNA inhibitor includes, without limitation, a miR-501-3p inhibitor, a miR-502-3p inhibitor, a miR-877-5p inhibitor, a miR-500a-3p inhibitor, a miR-664b-3p inhibitor, a miR-4508 inhibitor, a miR-1237-5p inhibitor, a miR-5001-5p inhibitor, a miR-4497 inhibitor, a miR-103-3p inhibitor, a miR-103a-3p inhibitor, a miR-124-3p inhibitor, a miR-24-3p inhibitor, a let-7a-5p inhibitor, a miR-185-5p inhibitor, a miR-320b inhibitor, a let-7d-5p inhibitor, a miR-140-3p inhibitor, a miR-17-5p inhibitor, a miR-151a-5p inhibitor, or combinations thereof.

In some embodiments, the administered inhibitor includes a miR-501-3p inhibitor. In some embodiments, the administered inhibitor includes a miR-502-3p inhibitor. In some embodiments, the administered inhibitor includes a miR-877-5p inhibitor.

The miRNA inhibitors of the present disclosure generally refer to molecules or sequences that can reduce or inhibit the activity of at least one miRNA of the present disclosure (i.e., a miRNA of any one of SEQ ID NOS: 1-30). In some embodiments, the miRNA inhibitor includes a reverse complement sequence of an miRNA to be inhibited. For instance, in some embodiments, a miR-502-3p inhibitor includes SEQ ID NO: 31, which represents a reverse complement sequence of miR-502-3p (SEQ ID NO: 2).

Administration of the miRNAs and/or miRNA Inhibitors

Various methods may be utilized to administer miRNAs and miRNA inhibitors to subjects. For instance, in some embodiments, the administration occurs by intravenous administration, subcutaneous administration, transdermal administration, topical administration, intraarterial administration, intrathecal administration, intracranial administration, intraperitoneal administration, intraspinal administration, intranasal administration, intraocular administration, oral administration, intratumor administration, or combinations thereof. In some embodiments, the administration occurs by intracranial administration.

### Neurological Disorders

The methods of the present disclosure may be utilized to treat or prevent various neurological disorders. For instance, in some embodiments, the neurological disorder includes, without limitation, Alzheimer's Disease (AD), Huntington's Disease, Parkinson's Disease, Amyotrophic Lateral Sclerosis (ALS), Multiple Sclerosis (MS), dementia, mild cognitive impairment (MCI), Schizophrenia, or combinations thereof. In some embodiments, the neurological disorder to be treated or prevented includes Alzheimer's Disease (AD).

The methods of the present disclosure can have various effects on a neurological disorder. For instance, in some embodiments, the methods of the present disclosure prevent a neurological disorder. In some embodiments, the methods of the present disclosure treat a neurological disorder. In some embodiments, the methods of the present disclosure treat and prevent a neurological disorder.

### Subjects

The methods of the present disclosure may be utilized to treat or prevent neurological disorders in various subjects. For instance, in some embodiments, the subject is a human being. In some embodiments, the subject is suffering from a neurological disorder. In some embodiments, the subject is vulnerable to a neurological disorder. In some embodiments, the subject exhibits symptoms of a neurological disorder.

### Compositions

Additional embodiments of the present disclosure pertain to compositions that include at least one microRNA (miRNA), at least one inhibitor of the miRNA, or combinations thereof. In some embodiments, the composition is suitable for use in treating or preventing a neurological disorder in a subject.

In some embodiments, the compositions of the present disclosure include at least one miRNA. In some embodiments, the miRNA in the composition includes, without limitation, miR-501-3p (SEQ ID NO: 1), miR-502-3p (SEQ ID NO: 2), miR-877-5p (SEQ ID NO: 3), miR-500a-3p (SEQ ID NO: 4), miR-664b-3p (SEQ ID NO: 5), miR-4508 (SEQ ID NO: 6), miR-1237-5p (SEQ ID NO: 7), miR-5001-5p (SEQ ID NO: 8), miR-4497 (SEQ ID NO: 9), miR-103-3p (SEQ ID NO: 10), miR-103a-3p (SEQ ID NO: 11), miR-124-3p (SEQ ID NO: 12), miR-24-3p (SEQ ID NO: 13), let-7a-5p (SEQ ID NO: 14), miR-185-5p (SEQ ID NO: 15), miR-320b (SEQ ID NO: 16), let-7d-5p (SEQ ID NO: 17), miR-140-3p (SEQ ID NO: 18), miR-17-5p (SEQ ID NO: 19), miR-151a-5p (SEQ ID NO: 20), miR-3196 (SEQ ID NO: 21), miR-6511b-5p (SEQ ID NO: 22), miR-4508 (SEQ ID NO: 23), miR-1237-3p (SEQ ID NO: 24), miR-5001-5p (SEQ ID NO: 25), miR-4492 (SEQ ID NO: 26), miR-4499 (SEQ ID NO: 27), miR-4497 (SEQ ID NO: 28), miR-107 (SEQ ID NO: 29), miR-138-5p (SEQ ID NO: 30), derivatives thereof, or combinations thereof. In some embodiments, the miRNA in the composition includes, without limitation, miR-3196 (SEQ ID NO: 21), miR-6511b-5p (SEQ ID NO: 22), miR-4508 (SEQ ID NO: 23), miR-1237-5p (SEQ ID NO: 7), miR-5001-5p (SEQ ID NO: 8), miR-4492 (SEQ ID NO: 26), miR-4499 (SEQ ID NO: 27), miR-4497 (SEQ ID NO: 28), miR 107 (SEQ ID NO: 29), derivatives thereof, or combinations thereof.

In some embodiments, the compositions of the present disclosure include a derivative of any one of the aforementioned miRNAs. In some embodiments, the miRNA derivative shares at least 70% sequence identity with any of the aforementioned miRNAs. In some embodiments, the miRNA derivative shares at least 75% sequence identity with any of the aforementioned miRNAs. In some embodiments, the miRNA derivative shares at least 80% sequence identity with any of the aforementioned miRNAs. In some embodiments, the miRNA derivative shares at least 85% sequence identity with any of the aforementioned miRNAs. In some embodiments, the miRNA derivative shares at least 90% sequence identity with any of the aforementioned miRNAs. In some embodiments, the miRNA derivative shares at least 95% sequence identity with any of the aforementioned miRNAs. In some embodiments, the miRNA derivative shares at least 99% sequence identity with any of the aforementioned miRNAs.

In some embodiments, the composition includes at least one miRNA inhibitor. In some embodiments, the miRNA inhibitor in the composition includes, without limitation, a miR-501-3p inhibitor, a miR-502-3p inhibitor, a miR-877-5p inhibitor, a miR-500a-3p inhibitor, a miR-664b-3p inhibitor, a miR-4508 inhibitor, a miR-1237-5p inhibitor, a miR-5001-5p inhibitor, a miR-4497 inhibitor, a miR-103-3p inhibitor, a miR-103a-3p inhibitor, a miR-124-3p inhibitor, a miR-24-3p inhibitor, a let-7a-5p inhibitor, a miR-185-5p inhibitor, a miR-320b inhibitor, a let-7d-5p inhibitor, a miR-140-3p inhibitor, a miR-17-5p inhibitor, a miR-151a-5p inhibitor, a miR-3196 inhibitor, a miR-651 lb-5p inhibitor, a miR-4508 inhibitor, a miR-1237-3p inhibitor, a miR-5001-5p inhibitor, a miR-4492 inhibitor, a miR-4499 inhibitor, a miR-4497 inhibitor, a miR-107 inhibitor, a miR-138-5p inhibitor, or combinations thereof.

In some embodiments, the miRNA inhibitor in the composition includes, without limitation, a miR-501-3p inhibitor, a miR-502-3p inhibitor, a miR-877-5p inhibitor, a miR-500a-3p inhibitor, a miR-664b-3p inhibitor, a miR-4508 inhibitor, a miR-1237-5p inhibitor, a miR-5001-5p inhibitor, a miR-4497 inhibitor, a miR-103-3p inhibitor, a miR-103a-3p inhibitor, a miR-124-3p inhibitor, a miR-24-3p inhibitor, a let-7a-5p inhibitor, a miR-185-5p inhibitor, a miR-320b inhibitor, a let-7d-5p inhibitor, a miR-140-3p inhibitor, a miR-17-5p inhibitor, a miR-151a-5p inhibitor, or combinations thereof.

In some embodiments, the miRNA inhibitor in the composition includes a miR-501-3p inhibitor. In some embodiments, the miRNA inhibitor in the composition includes a miR-502-3p inhibitor. In some embodiments, the miRNA inhibitor in the composition includes a miR-877-5p inhibitor. In some embodiments, the miRNA inhibitor in the composition includes a miR-500a-3p inhibitor.

miRNA inhibitors in the compositions of the present disclosure generally refer to molecules or sequences that can reduce or inhibit the activity of at least one miRNA of the present disclosure (i.e., a miRNA of any one of SEQ ID NOS: 1-30). In some embodiments, the miRNA inhibitor includes a reverse complement sequence of an miRNA to be inhibited. For instance, in some embodiments, a miR-502-3p inhibitor includes SEQ ID NO: 31, which represents a reverse complement sequence of miR-502-3p (SEQ ID NO: 2).

The compositions of the present disclosure can include various concentrations of miRNAs and miRNA inhibitors. For instance, in some embodiments, the compositions of the present disclosure include miRNA concentrations of at least 1 wt %. In some embodiments, the compositions of the present disclosure include miRNA concentrations of at least 5 wt %. In some embodiments, the compositions of the present disclosure include miRNA concentrations of at least 10 wt %. In some embodiments, the compositions of the present disclosure include miRNA inhibitor concentrations of at least 1 wt %. In some embodiments, the compositions of the present disclosure include miRNA inhibitor concentrations of at least 5 wt %. In some embodiments, the compositions of the present disclosure include miRNA inhibitor concentrations of at least 10 wt %.

The compositions of the present disclosure can include additional components. For instance, in some embodiments, the compositions of the present disclosure also include at least one pharmaceutically acceptable carrier. In some embodiments, the pharmaceutically acceptable carrier includes at least one excipient. In some embodiments, the excipient includes, without limitation, anti-adherents, binders, coatings, colors, disintegrants, flavors, glidants, lubricants, preservatives, sorbents, sweeteners, vehicles, or combinations thereof.

In some embodiments, the compositions of the present disclosure also include at least one solubilizing agent. In some embodiments, the solubilizing agent includes, without limitation, polyethylene glycol, glycerin, propylene glycol, ethanol, sorbitol, polyoxyethylated glycerides, polyoxyethylated oleic glycerides, polysorbates, sorbitan monooleate, hydroxypropyl-beta-cyclodextrin (HPCD), polyoxyl 40 hydrogenated castor oil, polyoxyl hydroxystearates, or combinations thereof.

The compositions of the present disclosure may be in various forms. For instance, in some embodiments, the compositions of the present disclosure may be associated with a delivery agent, such as a nanoparticle.

### ADDITIONAL EMBODIMENTS

Reference will now be made to more specific embodiments of the present disclosure and experimental results that provide support for such embodiments. However, Applicants note that the disclosure below is for illustrative purposes only and is not intended to limit the scope of the claimed subject matter in any way.

### Example 1. Synaptosome microRNAs Regulate Synapse Function in Alzheimer's Disease

MicroRNAs (miRNAs) are found in nerve terminals, synaptic vesicles, and synaptosomes. However, is unclear whether synaptic and cytosolic miRNA populations differ in Alzheimer's disease (AD) or if synaptosome miRNAs affect AD synapse activity. To address these questions, Applicant generated synaptosome and cytosolic fractions from postmortem brains of AD and unaffected control (UC) samples and analyzed them using a global Affymetrix miRNAs microarray platform. A group of miRNAs significantly differed (p<0.0001) with high fold changes variance (+/−>200-fold) in their expression in different comparisons—1) UC synaptosome vs UC cytosol, 2) AD synaptosome vs AD cytosol, 3) AD cytosol vs UC cytosol, and 4) AD synaptosome vs UC synaptosome. A validation analysis revealed miRNAs that were consistently different across sample groups. These miRNAs were further validated using brains of APP transgenic (Tg2576), Tau transgenic (P301L), and wild type mice. The miR-501-3p, miR-502-3p and miR-877-5p were identified as potential synaptosomal miRNAs upregulated with disease progression based on AD Braak stages. Gene Ontology Enrichment and Ingenuity Pathway Analysis of synaptosome miRNAs showed the involvement of these miRNAs in nervous system development, cell junction organization, synapse assembly formation, and function of GABAergic synapse. This is the first description of synaptic versus cytosolic miRNAs in AD.

miRNAs are present throughout cells and some miRNAs are localized to cellular organelles. Subcellular compartmentalization and localization of miRNAs, miRNA induced silencing complex and target mRNA have been observed to localize in multiple subcellular compartments, including the rough endoplasmic reticulum, processing (P)-bodies, stress granules the trans-Golgi network, early/late endosomes, multivesicular bodies, lysosomes and mitochondria.

Several studies identified the presence of miRNAs at the synapse and in synaptosomal fractions and determined their important roles in the regulation of local protein synthesis. Even synaptic vesicles extracted from mouse central nervous systems contain several small RNAs, transfer-RNAs and miRNAs. Additionally, miRNAs were found to be abundantly expressed within synaptoneurosomes isolated from prion-infected forebrain.

Since the 1980s, researchers began using synaptosomes prepared from postmortem brains to study AD-associated deficits in neurotransmission, including dysfunction of excitatory synapse acetylcholine, glutamate or aspartate, and inhibitory synapse glycine or (gamma-aminobutyric acid) GABA systems. A decrease in GABAergic synapse activity and inhibitory interneurons could contribute to AD progression and cognitive deficits in human and AD mouse models. Synaptic disturbances at the excitatory and inhibitory synapse in the forebrain have been found to contribute to the progression of AD and dementia. Recent synaptosomal studies have revealed decreased levels of neprilysin in AD patients. Neprilysin plays a key role in the clearance of AR.

Recently, it has been acknowledged that miRNAs exert widespread regulation over the translation and degradation of their target genes in the nervous system. Increasing evidence suggests that quite a few specific miRNAs play important roles in various aspects of synaptic plasticity, including synaptic activity, synaptic development, synaptogenesis, synaptic morphology, synaptic remodeling, synaptic scaling, synaptic excitability, synaptic ATP production and synaptic integrity. More importantly, the miRNA-mediated regulation of synaptic plasticity is not only responsible for synapse development and function but also involved in the pathophysiology of plasticity-related diseases, including AD.

MiRNAs are the potential regulators of genes and gene products and their therapeutic relevance has been explored in human diseases, including AD. The role of miRNAs has been exposed in the regulation of synaptic activity in the case of AD.

MiRNAs which enrich at synapse directly regulate the local protein synthesis involved in multiple synaptic functions and governing synaptic plasticity. However, the role of synaptosome-specific miRNAs is not determined in the progression of AD. In particular, there are no published reports about synaptosome-specific miRNAs for AD thus far. Also, it is not clear whether synaptosome miRNAs are different from cytosolic miRNAs.

Hence, this Example classified synaptosome versus cytosolic miRNAs and unfurled the possible molecular link of synaptosome miRNAs and AD progression. Applicant's study addressed four previously unknown important research questions—1) Are miRNA(s) levels altered at synaptosome in AD? 2) If so, are synapse miRNAs expressed differently in AD than in a healthy state? 3) Is synaptosome miRNAs expressed differentially in the cytosol? and 4) What function do synaptosome miRNAs play in synaptic activity and neurotransmission in AD? Overall, the focus of this Example was to discover the synaptosome miRNAs and understand their positive and negatives roles in AD progression.

### Example 1.1. Postmortem Brain Samples

Postmortem brains from AD patients and unaffected controls were obtained from several sources. Brain tissues were dissected from the Brodmann's Area 10 of the frontal cortices from AD patients (n=27) and age and sex matched unaffected controls (n=15).

### Example 1.2. Synaptosomes Extraction

Synaptosomes were extracted using Syn-PER Reagent as per manufacturer instructions with some modifications. Briefly, 50 mg of brain tissue was used from each sample for synaptosome extraction in 1 ml of Syn-PER Reagent. Tissues were homogenized slowly by Dounce glass homogenization on ice with ˜10 slow strokes. The resulting tissue homogenates were transferred to a centrifuge tube. Samples were centrifuged at 1400×g for 10 minutes at 4° C. to remove the leftover tissue debris. After centrifugation, the supernatant was transferred to a new tube. Again, supernatant (homogenate) was centrifuged at high speed 15,000×g for 20 minutes at 4° C. The supernatant was removed as cytosolic fraction and synaptosomes recovered in the pellet form. Both cytosolic fraction and synaptosome pellet were processed for RNA and protein extraction. Synaptosome pellet was also processed for transmission electron microscopic (TEM) analysis.

### Example 1.3. Synaptosomes Characterization

Synaptosome preparations (purity and integrity) were characterized by TEM analysis of synapse assembly, immunoblotting of synaptic proteins—synapse associate protein 25 (SNAP25), PSD95 and synaptophysin and qRT-PCR analysis of similar synaptic genes.

### Example 1.4. Transmission Electron Microscopy of Synaptosomes

Freshly isolated synaptosomes were processed for TEM analysis. Briefly, the pellet was fixed in a solution of 0.1M cacodylate buffer, 1.5% paraformaldehyde and 2.5% glutaraldehyde and then post-fixed with 1% osmium tetroxide and embedded in LX-112 resin. Ultrathin sections were cut, stained with uranyl acetate and lead citrate, and examined with the Hitachi H-7650/Transmission Electron Microscope at 60 kV. Low-magnification imaging was followed by high-magnification imaging. Representative images were acquired and recorded with an AMT digital camera.

### Example 1.5. Immunoblotting Analysis

Applicant performed immunoblot analysis for the synaptic/cytosolic proteins, brain cells and miRNAs biogenesis proteins. The 40 g of protein lysates were resolved on a 4-12% Nu-PAGE gel (Invitrogen). The resolved proteins were transferred to nylon membranes (Novax Inc., San Diego, CA, USA) and then incubated for 1 h at room temperature with a blocking buffer (5% dry milk dissolved in a TBST buffer). The nylon membranes were incubated overnight with the primary antibodies. The membranes were washed with a TBST buffer 3 times at 10-min intervals and then incubated for 2 h with an appropriate secondary antibody, sheep anti-mouse HRP 1:10,000, followed by three additional washes at 10-min intervals. Proteins were detected with chemiluminescence reagents (Pierce Biotechnology, Rockford, IL, USA), and the bands from the immunoblots were visualized.

### Example 1.6. Quantitative Real-Time PCR Analysis

Quantification of mRNA levels of synaptic genes was carried out with real-time qRT-PCR. The oligonucleotide primers were designed with primer express software (Applied Biosystems) for SNAP25, synaptophysin, PSD95, elF1a and PCNA. SYBR-Green chemistry-based quantitative real-time qRT-PCR was used to measure mRNA expression of these genes using β-actin as housekeeping genes.

### Example 1.7. Affymetrix miRNA Microarray Analysis

Initially, Applicant used five AD postmortem and five unaffected control postmortem brains for Affymetrix microarray analysis. Total RNA was extracted from the synaptosomal and cytosolic fractions from both AD and health control samples using the TriZol reagent with some modifications. In total, Applicant had 20 samples for miRNA analysis—AD synaptosome (n=5), UC synaptosome (n=5), AD cytosol (n=5) and UC cytosol (n=5). The miRNA expression profiles were generated with Affymetrix GeneChip miRNA array v. 4.0 (Supplementary information).

### Example 1.8. Microarray Data Analysis

Data was analyzed using four comparisons—1) AD synaptosome vs AD cytosol, 2) unaffected control (UC) synaptosome vs UC cytosol, 3) AD cytosol vs UC cytosol, and 4) AD synaptosome vs UC synaptosome. Microarray data for miRNAs expression changes in synaptosomal vs cytosol fractions were analyzed using two main criteria—Gene-level fold change <−2 or >2 and Gene-level P-value <0.05. A probe set (Gene/Exon) is considered expressed if ≥50% samples have detectable above background (DABG) values below DABG Threshold <0.05.

### Example 1.9. Validation of Deregulated miRNAs Using Postmortem Brains

The deregulated miRNAs obtained from Affymetrix analysis were further tested and validated on large number of AD postmortem brains (n=27) and unaffected controls (n=15). Validation of miRNAs were performed for four comparisons—1) AD synaptosome vs AD cytosol, 2) Unaffected control (UC) synaptosome vs UC cytosol, 3) AD cytosol vs UC cytosol, and 4) AD synaptosome vs UC synaptosome. MiRNAs levels were quantified by using miRNAs qRT-PCR, which involved three steps (i) miRNAs polyadenylation, (ii) cDNA synthesis and (iii) qRT-PCR. Primers for desired miRNAs were synthesized commercially (Integrated DNA Technologies Inc., IA, USA). To normalize the miRNA expression, U6 snRNA and sno-202 were used as internal controls. The reaction mixture of each sample was prepared in triplicates. The reaction was set in the 7900HT Fast Real Time PCR System (Applied Biosystems, USA). qRT-PCR was performed in triplicate, and the data were expressed as the mean±SD.

### Example 1.10. Validation of Differentially Expressed miRNAs Using AD Mouse Models

The deregulated miRNAs obtained from Affymetrix analysis were further validated using brain tissues from 12 months old APP Transgenic (Tg2576) (n=6), Tau transgenic (P301L) (n=7) and age and sex matched wild type (WT) (n=8) mice. The deregulated miRNAs were conserved in both human and mice. The APP Tg, Tau Tg and WT mice were obtained from Jackson Laboratories and colonies were maintained in Applicant's lab. Mice were euthanized to extract the brain tissues. The brains were dissected, and the cerebral cortex was used for cytosol and synaptosome miRNA extraction. Validation of miRNAs were performed for four comparisons—1) AD mice synaptosome vs cytosol, 2) WT mice synaptosome vs cytosol, 3) AD mice cytosol vs WT mice cytosol, and 4) AD mice synaptosome vs WT mice synaptosome. MiRNAs levels in APP and Tau mice relative to WT mice were quantified by using miRNAs qRT-PCR.

### Example 1.11. In-Silico Analysis for Potential miRNAs

The QIAGEN's Ingenuity® Pathway Analysis (IPA®, QIAGEN Inc., https://www.qiagenbioinformatics.com/products/ingenuity-pathway-analysis) program was used to analyze the synaptosome and cytosolic miRNAs target genes with FDR p-values <0.05 and with p-value <0.05. The IPA was used to gain insight into the overall biological changes caused by the expression, target gene prediction for synaptosome and cytosolic miRNAs with AD and unaffected controls and gene Integrated Analysis. Each gene was related to various functions, pathways, and diseases as analyzed by using Ingenuity knowledge base platform. The miRNA target genes (predicted and validated) were identified using various on-line miRNA algorithms.

### Example 1.12. Statistical Considerations

Statistical parameters were calculated using Prism software, v6 (La Zolla, CA, USA). Results are reported as mean±SD. The results were analyzed by two-tailed Student's t-test to evaluate miRNAs expression in two groups of samples—1) AD synaptosome vs AD cytosol, 2) unaffected control (UC) synaptosome vs UC cytosol, 3) AD cytosol vs HC cytosol, and 4) AD synaptosome vs UC synaptosome. One-way comparative analysis of variance was used for analyzing WT, APP Tg and Tau Tg mice synaptosome vs cytosolic miRNAs data. Significant differences in three group of samples were calculated by Bonferroni's multiple comparison tests. The correlation of miRNAs fold changes with Braak stages were analyzed by Tukey's multiple comparisons test. P<0.05 was considered statistically significant.

### Example 1.13. Synaptosomes Preparations from Postmortem Brains

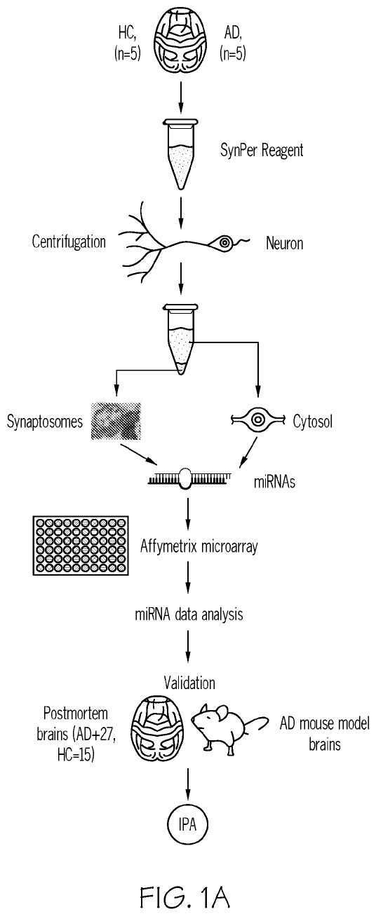

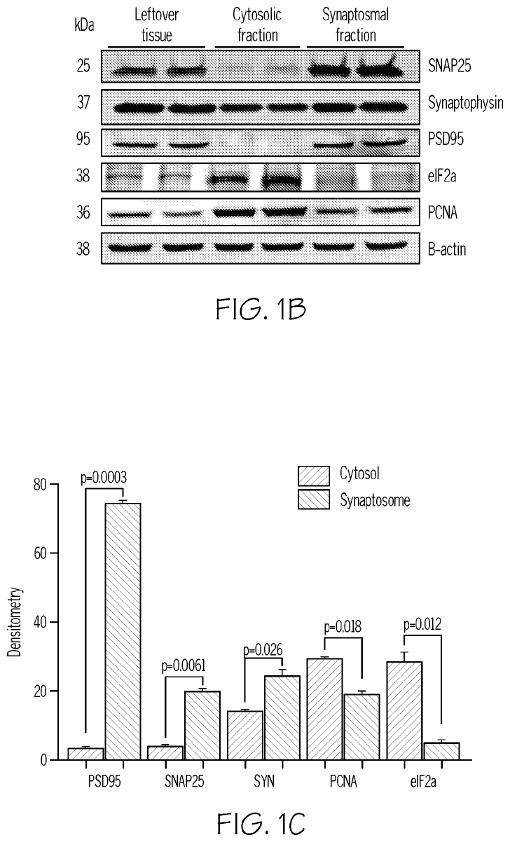

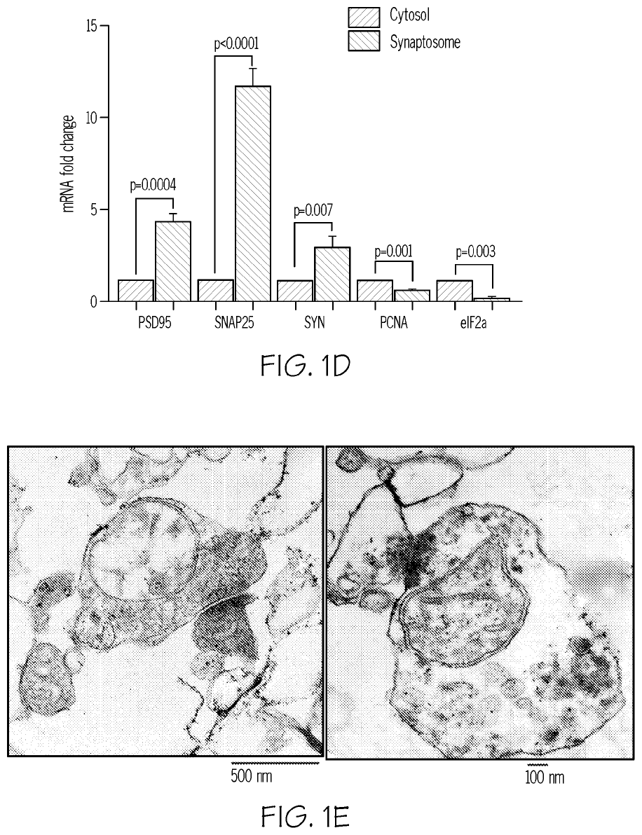

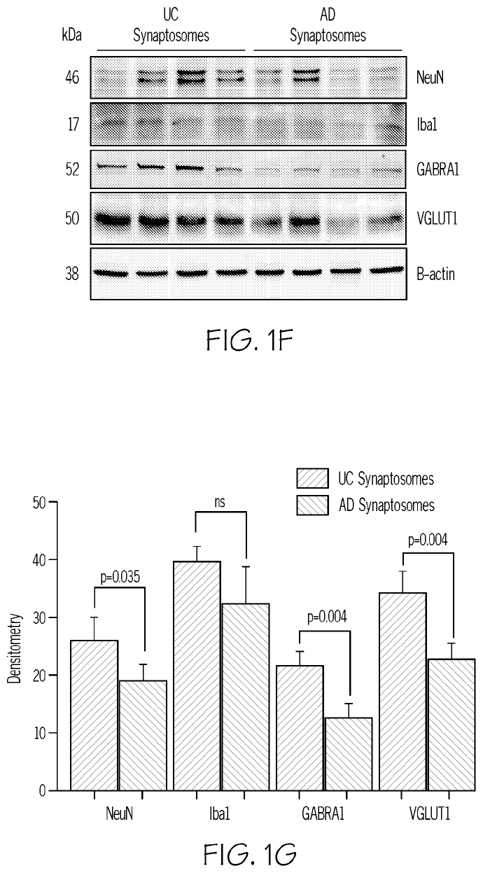

The increased levels of APP and Tau proteins were detected in AD cases compared to UC samples. Next, these samples were processed for synaptosome preparation downstream applications (FIG. 1A). To confirm the synaptosome fractionation, Applicant first processed the UC postmortem brains and prepared the protein lysates from cytosolic fraction, synaptosomal fractions and leftover tissue extract for synaptic and cytosolic protein analysis. FIG. 1B showed the immunoblots for synapse associated protein 25 (SNAP25), synaptophysin and PSD95 and cytosolic/nuclear proteins elF1a and PCNA. Densitometry analysis showed the significantly high levels of SNAP25, synaptophysin and PSD95 in the synaptosome fraction and reduced levels in cytosol (FIG. 1C). SNAP25 and PSD95 were completely absent from the cytosolic fraction, however synaptophysin was detected in the cytosolic fraction also as reported by other researchers. On the other hand, elF1a and PCNA protein levels were higher in cytosol. qRT PCR analysis also showed the increased expression of SNAP25, synaptophysin and PSD95 genes in the synaptosomes relative to cytosol and reduced expressions of elF1a and PCNA in the synaptosome fraction relative to cytosol (FIG. 1D). These results confirm a precise separation of cytosolic and synaptosomes fractions.

Next, Applicant processed the synaptosome fraction from AD patients and UC for TEM analysis (FIG. 1E). The electron micrograph revealed the distinct synapse assembly and intact synaptosomes with all the components—mitochondria, synaptic vesicles, endosomes, post-synaptic density protein and synaptic cleft. The mitochondrial structure and synaptic cleft were found to be distorted in AD postmortem brains while it was intact in control samples. These results confirmed the purity and integrity of synapse and synaptosome fraction.

Further, to confirm the brain cells specificity of synaptosome, Applicant checked the levels of cell type markers (NeuN—Neuron, Iba1—Microglia, GFAP—Astrocytes). Applicant found the significantly detectable levels of NeuN and Iba1 proteins (but not GFAP) in both UC and AD synaptosomes (FIG. 1F). NeuN level was found to be significantly reduced (p=0.035) in AD synaptosmes relative to UC synaptosomes (FIG. 1G). Applicant did not see any significant difference in Iba1 levels in AD vs UC synaptosome. These observations confirm the neuron specificity of synaptosomes.

Applicant also characterized the synaptosomes as excitatory or inhibitory based on the levels of excitatory and inhibitory synapse markers (VGLUT1 and GABRA1). Immunoblots in FIG. 1F showed the levels of both markers in UC and AD synaptosomes. The levels of VGLUT1 (p=0.004) and GABRA1 (p=0.004) proteins were significantly reduced in AD synaptosome relative to UC synaptosomes (FIG. 1G). These observations confirmed the presence of both types of synapses in synaptosomes fraction with their reduction in AD brains.

### Example 1.14. MicroRNAs Expression in UC Synaptosome Vs UC Cytosol

The miRNA microarray data of synaptosomal and cytosolic fractions were analyzed by Transcription analysis console v.4. A total of 43 mature miRNAs were found to be deregulated in UC synaptosomal fraction relative to UC cytosolic fraction. The 20 Homo sapiens (hsa) miRNAs were highly expressed in the synaptosomes and low in the cytosol. These observations indicate that highly expressed miRNAs in synaptosomes have functional importance of synaptosomal function.

The 23 hsa-miRNAs were highly expressed in the cytosol and showed reduced expression in the synaptosomes, strongly suggesting that these miRNAs have cytosolic relevance in healthy state. MiRNAs were characterized on several selection criteria-fold change, standard deviation, p-values, expression priority, transcript ID, chromosome location, strand specificity, start and stop codon, targeted and validated gene symbols.

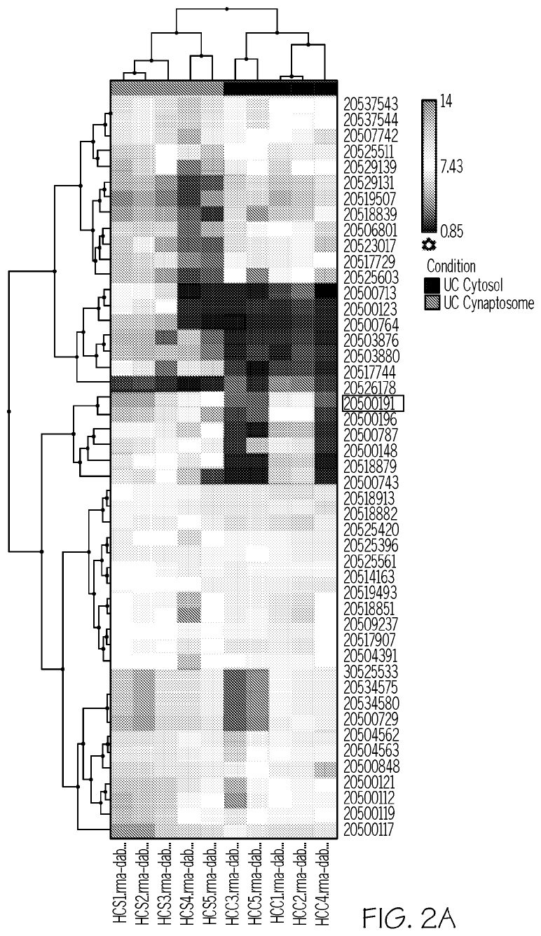

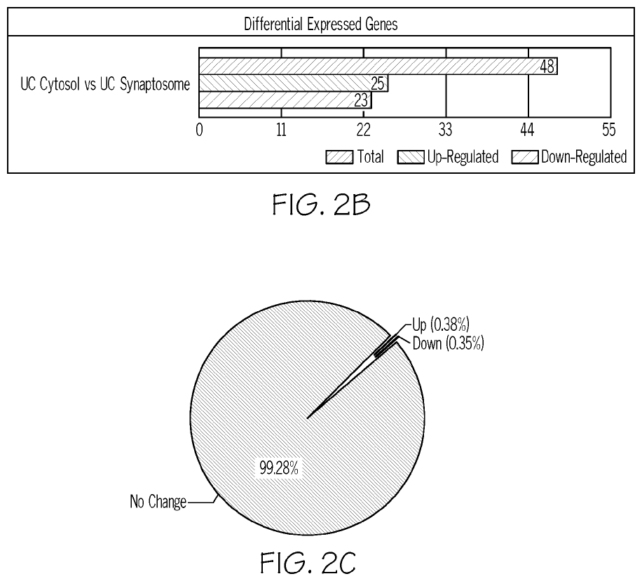

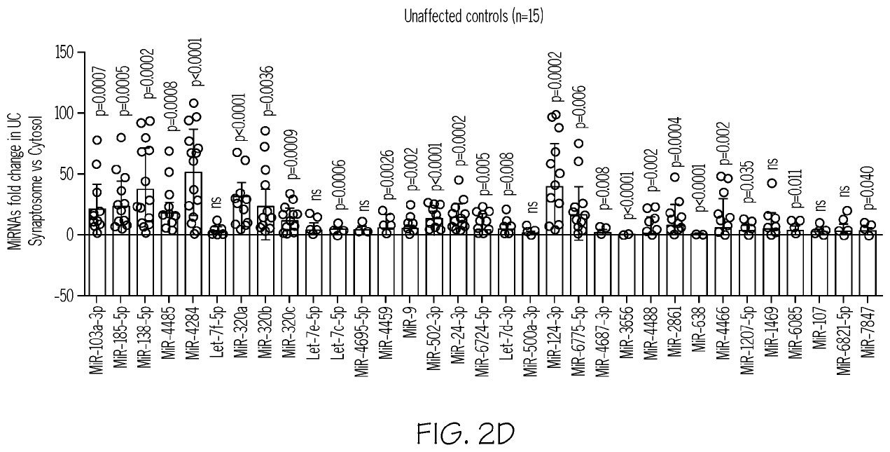

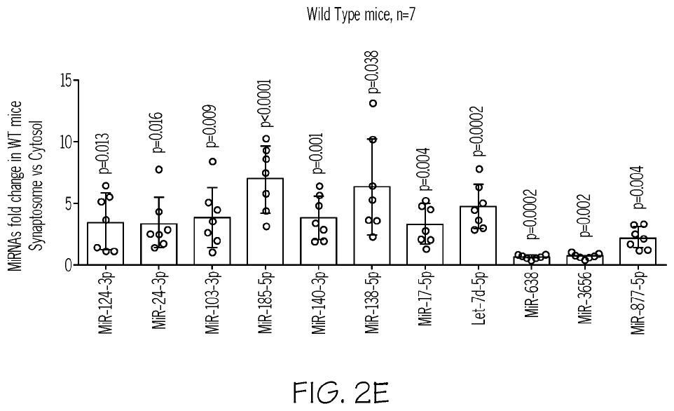

FIG. 2A shows the hierarchical clustering and heat map of significantly deregulated miRNAs with their ID numbers. As a result, 25 miRNAs were upregulated, and 23 miRNAs were downregulated significantly (FIG. 2B). Gene filter analysis of total miRNAs pool shows that 99.28% of miRNA population did not show significant difference in the cytosol vs synaptosome compartments. Only 0.38% population of miRNAs is upregulated, and 0.35% miRNA population is downregulated (FIG. 2C). A scattered plot showed the average log 2 fold changes values of significantly deregulated miRNAs and a volcano plot showed the p values (−log 10) of significantly deregulated miRNAs. The top candidate miRNAs were selected for validation analysis.

### Example 1.15. Validation Analysis of Synaptosome and Cytosolic miRNAs in Healthy State

UC postmortem brains. Validation analysis was performed on UC (n=15) postmortem brains to distinguish synaptosome and cytosolic miRNAs in the normal state. Out of the 43 deregulated miRNAs, only 33 miRNAs were successfully amplified by qRT-PCR using specific primers. The 18 miRNAs showed similar expression trends as obtained by Affymetrix data analysis. The remaining miRNAs either showed opposite trends to Affymetrix data or did not change significantly. Overall, 24 miRNAs were significantly upregulated in the synaptosomes relative to the cytosol and two miRNAs (miR-638 and miR-3656) were significantly downregulated in the synaptosomal fractions relative to the cytosolic fractions. Seven miRNAs did not show any significant changes (FIG. 2D).

Wildtype (WT) mice brains. Further, Applicant performed expression analysis of above classified synaptosome and cytosolic miRNAs in WT mice (n=7). A total of 11 Mus musculus (mmu)-miRNAs were amplified, and out of them, nine were significantly upregulated and two were downregulated in WT mice synaptosome relative to cytosol (FIG. 2E). The 11 miRNAs showed similar expression pattern as observed by primary screening and UC postmortem brain validation. Based on these observation nine miRNAs were classified as synaptosome miRNAs and two miRNAs as cytosolic miRNAs in the healthy state.

### Example 1.16. MicroRNAs Expression in AD Synaptosome Vs AD Cytosol

Next, Applicant compared the microarray data for miRNAs expression changes in AD synaptosomal fractions vs AD cytosolic fractions. A total of 39 mature miRNAs were found to be deregulated in AD synaptosome vs AD cytosol comparison and 28 hsa-miRNAs were highly expressed in the synaptosomes and low in the cytosol. The 11 out 39 miRNAs were highly expressed in the cytosol and showed reduced expression in the synaptosomes. The differential expression of these miRNAs in the AD synaptosomes and AD cytosol suggests their relevance in diseased state.

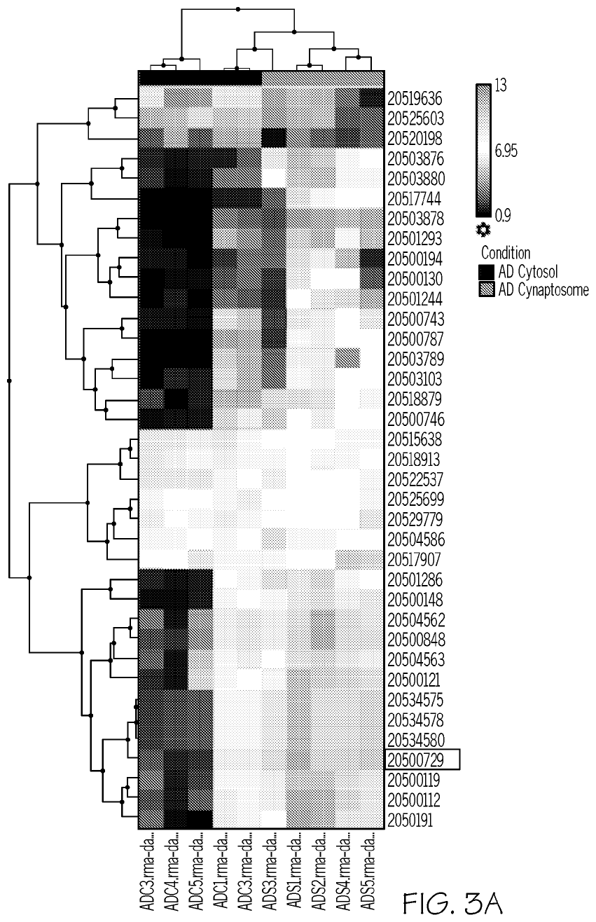

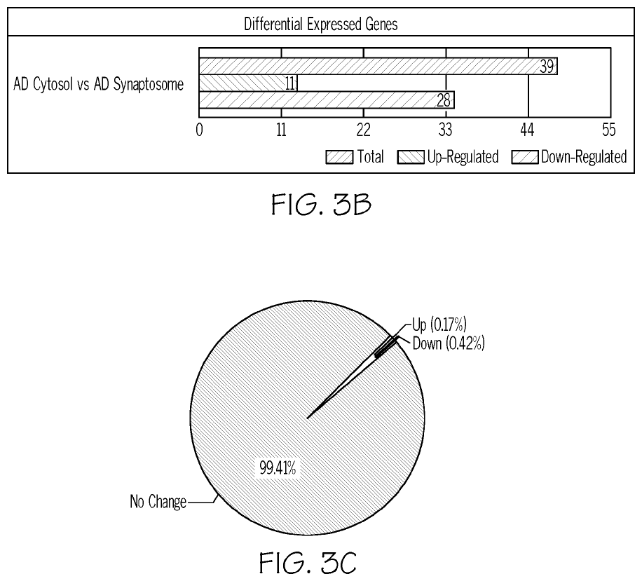

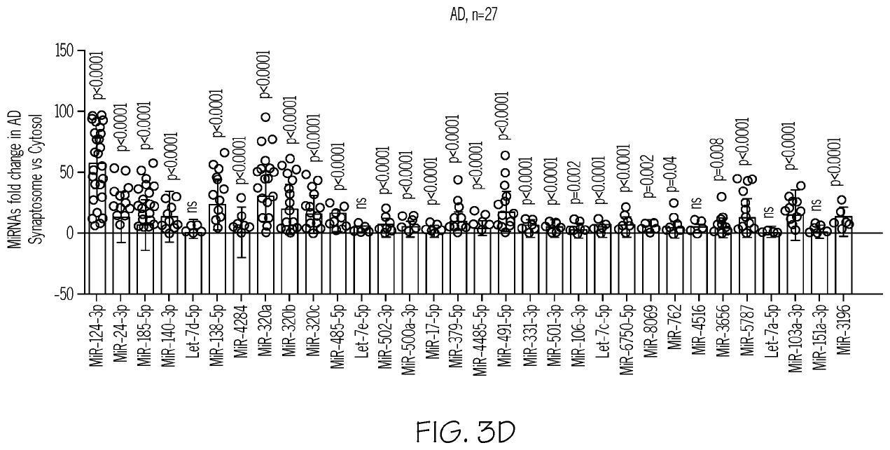

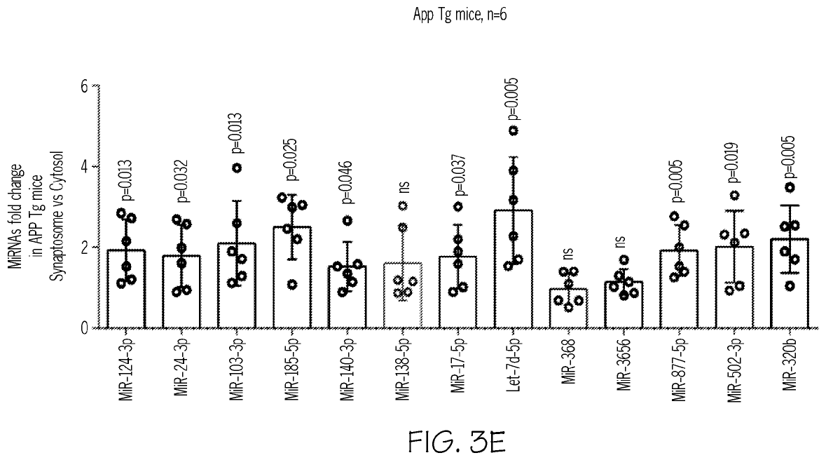

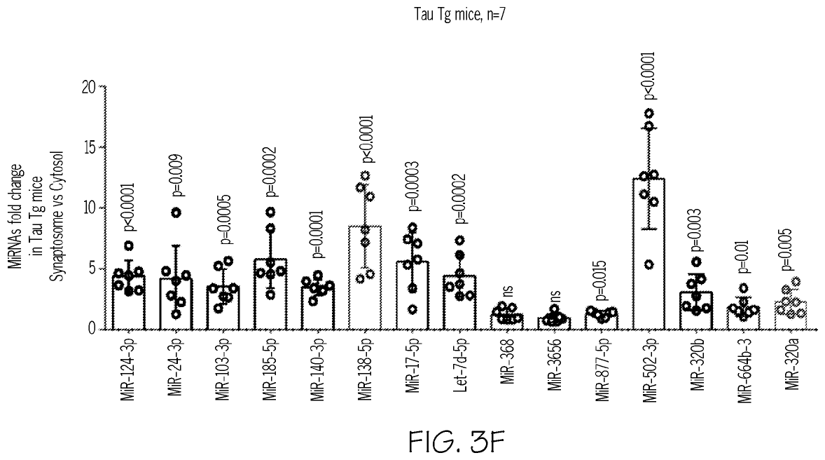

FIG. 3A shows hierarchical clustering and a heat map of significantly deregulated miRNAs with their ID numbers. The 11 miRNAs were upregulated in the cytosol and 28 miRNAs were downregulated in the cytosol significantly (FIG. 3B). Gene filter analysis of total miRNAs pool shows that 99.41% of miRNA population did not show significant difference in the cytosol vs synaptosome compartment, only, 0.59% of populations showed variable expression levels. The 0.17% of miRNAs are upregulated and 0.42% of miRNAs population is downregulated (FIG. 3C). A scattered plot showed the average log 2 fold changes values of significantly deregulated miRNAs and a volcano plot showed the p values (−log 10) of significantly deregulated miRNAs in AD synaptosome vs AD cytosol. Based on the miRNA(s) expression pattern in unaffected controls and AD samples, 22 miRNAs (37.3%) were expressed only in UC samples and 21 miRNAs (35.6%) were expressed only in AD samples. However, 16 miRNAs (27.1%) were commonly expressed in both conditions.

### Example 1.17. Validation Analysis of Synaptosome and Cytosolic miRNAs in AD State

AD postmortem brains. The top candidate miRNAs were selected for validation analysis. Validation analyses were performed on 27 AD postmortem brains to distinguish synaptosome and cytosolic miRNAs in the diseased state. Out of the 39 deregulated miRNAs, only 32 miRNAs were amplified by using specific primers. The 22 miRNAs showed the similar expression trend as obtained by Affymetrix data analysis. The remaining miRNAs were either showed opposite trend to Affymetrix data or did not change significantly. Overall, 27 miRNAs were significantly upregulated in the synaptosomes relative to the cytosol and no miRNA showed any significantly downregulation. The five miRNAs did not show any significant changes in the synaptosomes relative to the cytosol (FIG. 3D).

APP Tg mice. Next, Applicant performed synaptosome and cytosolic miRNAs validation using APP Tg mice (n=6). The 13 mmu-miRNAs showed similar expression pattern as observed by primary screening and AD postmortem brain validation. MiR-103-3p, miR-185-5p, miR-24-3p, miR-502-3p, miR-320b, let-7d-5p, miR-124-3p, miR-140-3p, miR-17-5p, and miR-877-5p showed significant upregulation in the synaptosome, while miR-138-5p, miR-3656 and miR-638 did not show any significantly changes in their expression (FIG. 3E).

Tau Tg mice. Further, Applicant performed synaptosome and cytosolic miRNAs validation using Tau Tg mice (n=7). The 13 mmu-miRNAs showed similar expression pattern as observed by primary screening and AD postmortem brain validation. MiR-103-3p, miR-185-5p, miR-24-3p, miR-502-3p, miR-320b, let-7d-5p, miR-124-3p, miR-140-3p, miR-17-5p, miR-877-5p, miR-320a and miR-664a-3p showed significant upregulation in the synaptosome, while miR-138-5p, miR-3656 and miR-638 did not show any significantly changes in their expression (FIG. 3F). Based on these observations, 11 miRNAs were classified as synaptosome miRNAs and two miRNAs as cytosolic miRNAs in the AD state.

### Example 1.18. MicroRNAs Expression in AD Cytosol Vs UC Cytosol

Next, Applicant compared AD cytosolic vs UC cytosolic miRNAs. A total of 13 hsa-miRNAs were found to be significantly deregulated in the AD cytosol vs UC cytosol comparison. Interestingly, expression levels of all miRNAs were reduced in AD cytosol. Moreover, hierarchical clustering and heat maps showed significantly deregulated miRNAs with their ID numbers. The 13 miRNAs were found to be downregulated significantly.

Gene filter analysis of total miRNAs pool showed that 99.76% of miRNA population did not show significant difference in the cytosol vs synaptosome compartment. Only 0.24% of miRNA populations showed variable expression levels. All 0.24% miRNA population is downregulated. A scattered plot showed the average log 2 fold changes values of significantly deregulated miRNAs and a volcano plot showed the p values (−log 10) of significantly deregulated miRNAs in AD cytosol vs AD cytosol. The top candidate miRNAs were selected for validation analysis.

### Example 1.19. Validation Analysis of Cytosolic miRNAs in AD and Unaffected Control

AD and UC postmortem brains. Validation analysis of cytosolic miRNAs were performed on 15 UC and 27 AD postmortem brain samples. The 13 miRNAs candidates were selected for validation analysis. Opposed to the Affymetrix data, nine miRNAs were significantly upregulated in AD cytosol relative to UC cytosol and three miRNAs did not show significant changes.

WT, APP Tg and Tau Tg mice. Applicant also performed the validation of cytosolic miRNAs in APP Tg and Tau Tg mice relative to WT mice. Other than the 13 cytosolic mmu-miRNAs, Applicant also checked the expression of other potential mmu-miRNAs: miR-17-5p, let-7d-5p, miR-185-5p, miR-103-3p, miR-138-5p, miR-877-5p, miR-24-3p, miR-502-3p, miR-140-3p, miR-124-3p and miR-3656. Most of the miRNAs were upregulated in the APP Tg and Tau Tg cytosol relative to WT cytosol. Only, miR-638 and miR-3656 were significantly down regulated in APP Tg cytosol relative to WT.

### Example 1.20. MicroRNAs Expression in AD Synaptosome Vs UC Synaptosome

Lastly, Applicant compared the microarray data for miRNAs expression changes in AD synaptosomes vs UC synaptosomes. A total of 11 miRNAs were found to be deregulated significantly in AD Synaptosomes vs UC synaptosomes comparison. Four hsa-miRNAs—miR-502-3p, miR-500a-3p, miR-877-5p and miR-664b-3p were highly expressed in AD synaptosomes relative to UC synaptosomes. The remaining seven hsa-miRNAs—miR-3196, miR-651 lb-5p, miR-4508, miR-1237-5p, miR-5001-5p, miR-4492 and miR-4497 showed reduced expression AD synaptosomes and highly expressed in UC synaptosomes. The differential expression of these miRNAs in AD and UC synaptosomes suggests their importance in synapse function.

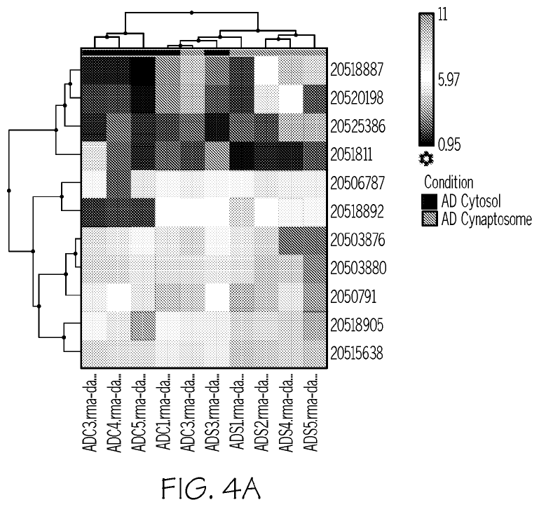

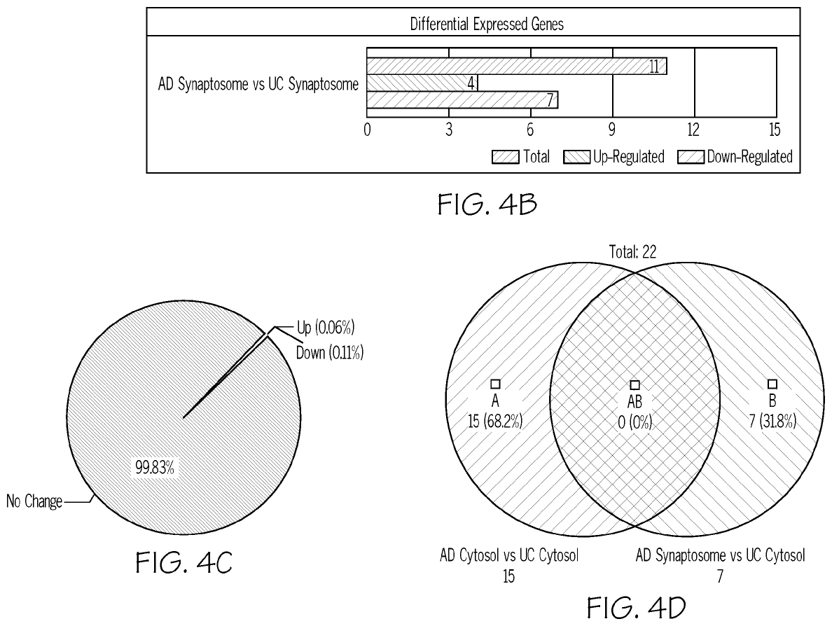

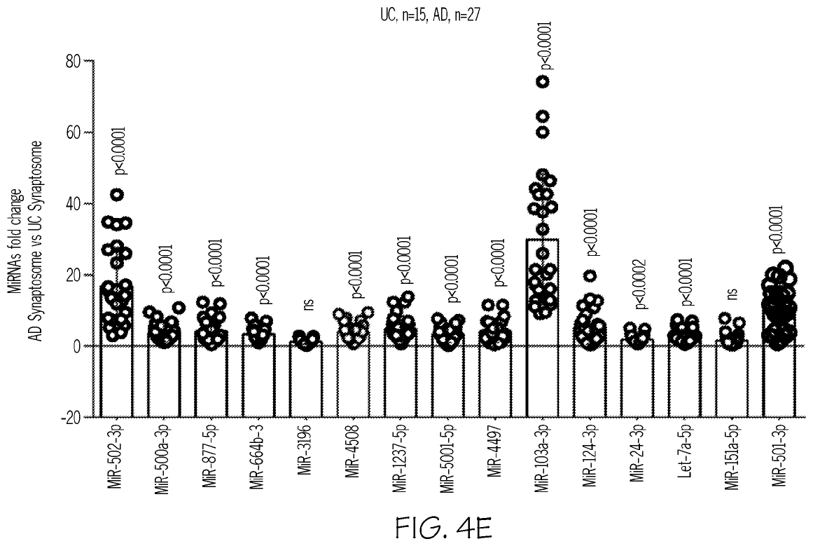

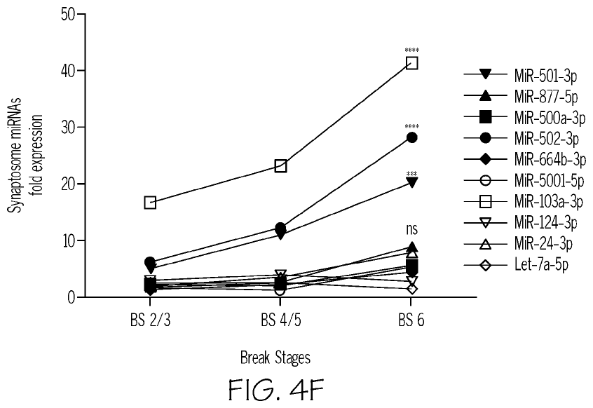

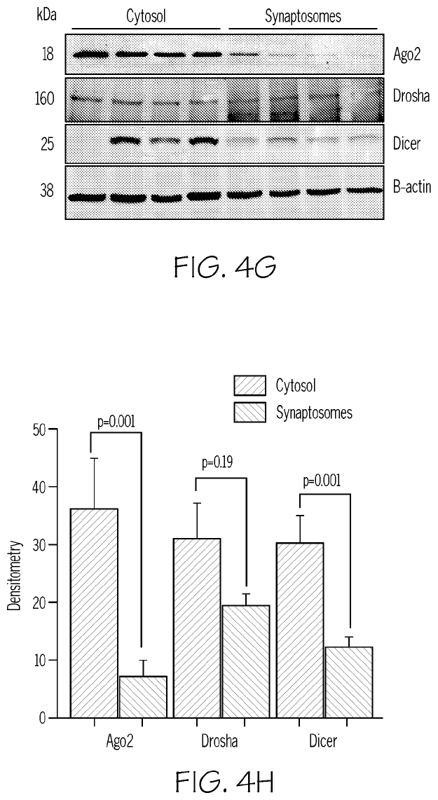

FIG. 4A showed the hierarchical clustering and heat map of significantly deregulated miRNAs with their ID numbers. The four miRNAs were upregulated, and seven miRNAs were downregulated significantly (FIG. 4B). Gene filter analysis of total miRNAs pool showed that 99.83% of the miRNA population did not show significant difference in the synaptosome compartments in AD vs UC. Only 0.17% miRNAs populations showed variable expression pattern. The 0.06% of miRNAs is upregulated and 0.11% of the miRNA population is downregulated (FIG. 4C). A scattered plot showed the average log 2 fold changes values of significantly deregulated miRNAs and a volcano plot showed the p values (−log 10) of significantly deregulated miRNAs in AD synaptosome vs UC synaptosomes. The top candidate miRNAs were selected for validation analysis.

Based on the miRNAs' expression pattern in cytosol and synaptosome in AD vs UC samples, 15 miRNAs (68.2%) were expressed only in the cytosol and seven miRNAs (31.8%) were expressed only in the synaptosomes. Applicant did not see any miRNA that were commonly expressed in both conditions (FIG. 4D).

### Example 1.21. Validation Analysis of Synaptosomal miRNAs

AD and UC postmortem brains. Validation analyses were performed on 15 UC and 27 AD postmortem brains. Applicant checked synaptosome expression of the most significantly deregulated 16 miRNAs. However, only 14 hsa-miRNAs were amplified, the 12 hsa-miRNAs (miR-502-3p, miR-500a-3p, miR-877-5p, miR-664b-3p, miR-4508, miR-1237-5p, miR-5001-5p, miR-4497, miR-103a-3p, miR-124-3p, miR-24-3p and let-7a-5p were significantly upregulated in the AD synaptosomes relative to UC synaptosomes, while two hsa-miRNAs (miR-3196 and miR-151-5p) did not show any significant changes (FIG. 4E).

WT, APP Tg and Tau Tg mice. Applicant also performed the validation of above-mentioned miRNAs and other potential synaptosome miRNAs in APP Tg and Tau Tg mice relative to WT mice. The 12 mmu-miRNAs, which were, amplified successfully included—miR-17-5p, let-7d-5p, miR-185-5p, miR-103-3p, miR-138-5p, miR-877-5p, miR-24-3p, miR-502-3p, miR-140-3p, miR-124-3p, miR-638 and miR-3656. In APP Tg mice synaptosomes seven miRNAs were significantly upregulated, four were significantly downregulated relative to WT synaptosomes and one miRNA showed no change. In Tau Tg synaptosomes, nine miRNAs were significantly upregulated, and three miRNAs were significantly downregulated relative to WT synaptosomes.

Summarizing all validation analyses, 12 miRNAs expression was consistent in different comparisons and samples settings. The 10 miRNAs can be classified as synaptosome miRNAs and two miRNAs as cytosolic miRNAs.

Next, Applicant examined the synaptosomal miRNAs expression patterns with AD samples Braak stages. Multiple comparison analysis showed that expression of synaptosomal miRNAs were gradually increased with Braak stages. However, significant differences were found in miR-501-3p (p=0.001), miR-502-3p (p<0.0001), miR-877-5p (p=0.01) and miR-103a-3p (p<0.0001) fold changes at Braak stage 6 relative to Braak stage 2/3 (FIG. 4F). These results unveiled the strong connection of these miRNAs with AD progression.

Further, to determine the synaptosomal miRNAs synthesis at synapse, Applicant checked the levels of key miRNA biogenesis proteins (Ago2, Drosha and Dicer) in cytosol and synaptosome fractions. In FIG. 4G, immunoblots showed the levels of miRNA biogenesis proteins in UC cytosol and synaptosomes. Densitometry analysis showed very high levels of all three proteins in cytosol relative to synaptosomes (FIG. 4H). The presence of miRNA biogenesis proteins in synaptosomes confirmed that miRNAs might be synthesize at synapse.

### Example 1.22. In Silico Ingenuity® Pathway Analysis of Cytosolic and Synaptosome miRNAs in AD and Healthy State

The deregulated miRNAs under different conditions were run for IPA analysis. The first comparison was cytosolic vs synaptosome miRNAs in the healthy state. The top deregulated miRNAs were involved in several diseases, molecular and cellular functions, physiological system development and functions.

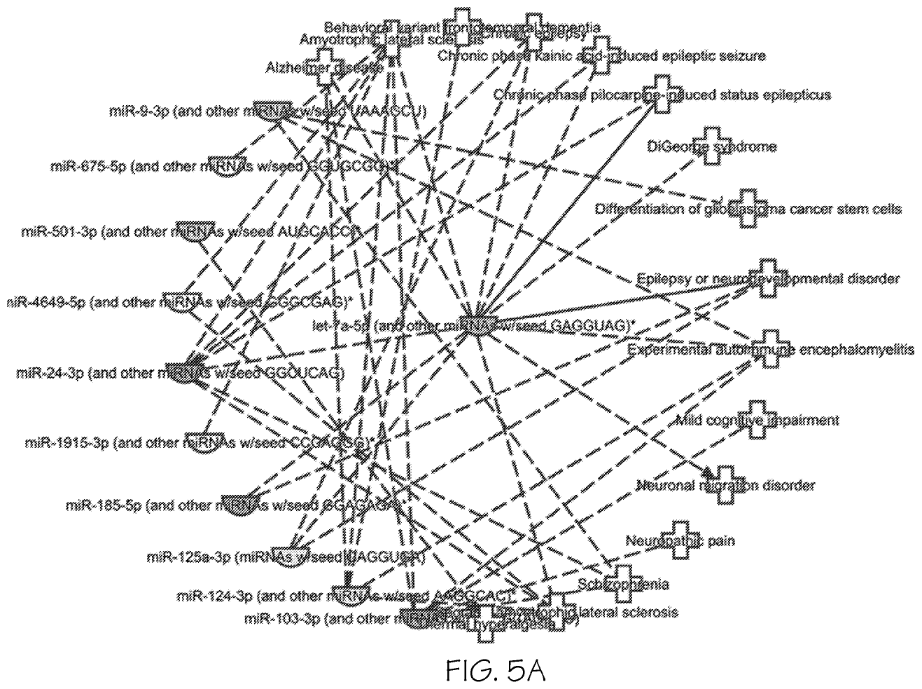

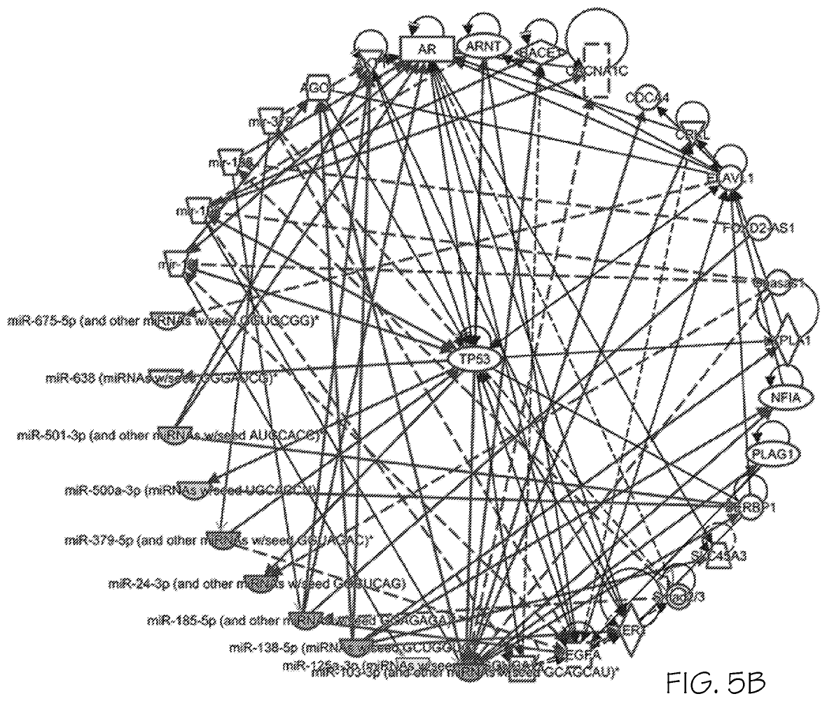

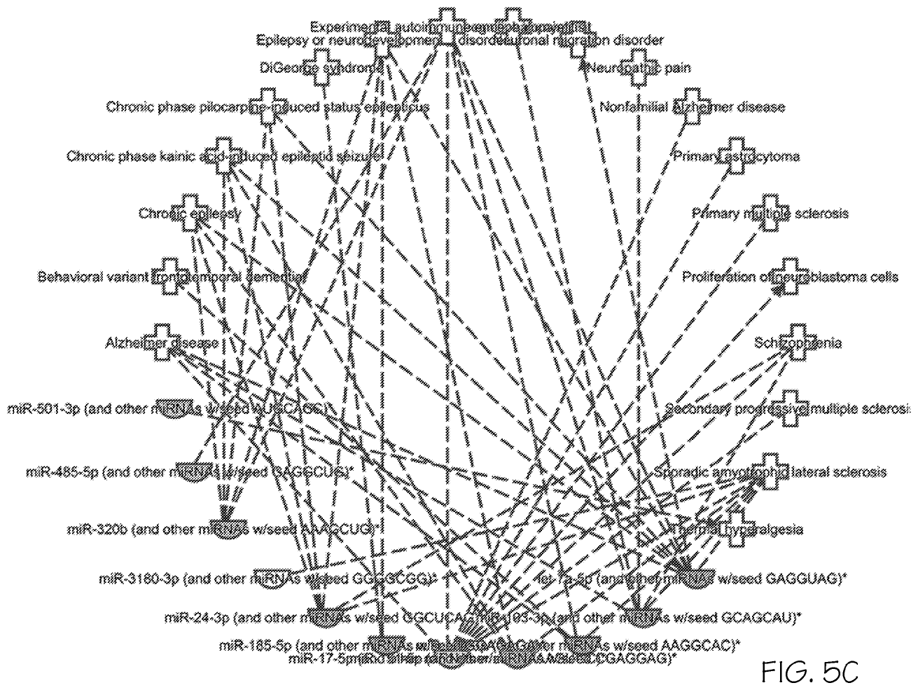

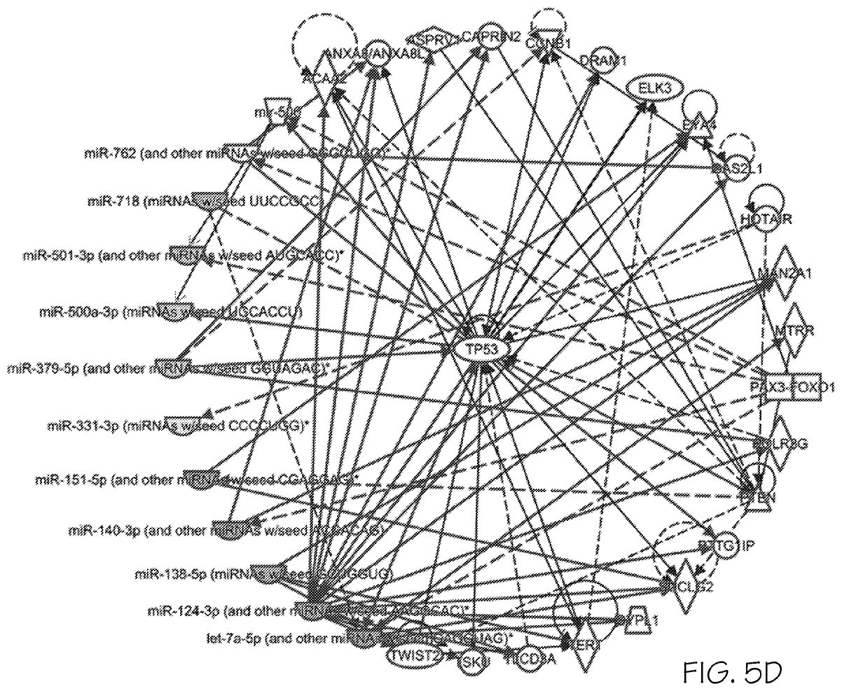

However, Applicant focused on the miRNA candidates, which are involved in nervous system development, function and neurological diseases. Eleven miRNAs were identified, which were significantly (P<0.05) involved in many neurological diseases and dementia, including AD and MCI (FIG. 5A). Next, Applicant analyzed the mRNA target and seed sequences of these miRNAs to understand the molecular mechanism of miRNAs involved in neurological function (FIG. 5B).

The tumor suppressor gene (TP53) was the central gene that was targeted by many of these miRNAs. Other potential genes were BACE1, Smad2/3, Lypla1, Akt1 and SERBP1 pathway genes. Similarly, Applicant studied synaptosome and cytosolic miRNAs function in AD cases. The top miRNA candidates were significantly (P<0.05) involved in several nervous system development, function and neurological diseases. However, Applicant's interest was neurological disorders and dementia, where eight miRNAs were detected which were involved in several neurological disorders including AD (FIG. 5C).

Further, miRNAs target predication analysis showed more than 20 genes that are targeted by these miRNAs (FIG. 5D). Next, Applicant studied the biological roles of cytosolic miRNAs which were downregulated in AD compared to UC. The top five miRNAs were significantly involved in several diseases and molecular pathways. MiRNAs and diseased pathways showed integration with Amyotrophic lateral sclerosis. Like other miRNAs, several genes were identified as potential target for these four miRNAs.

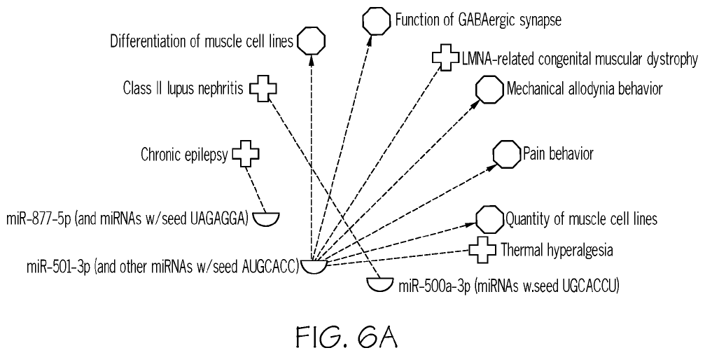

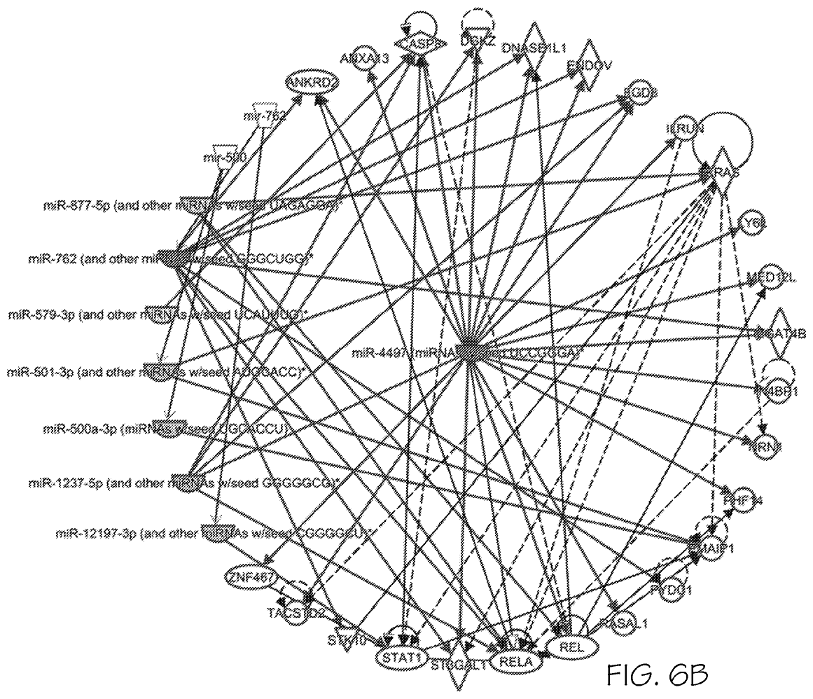

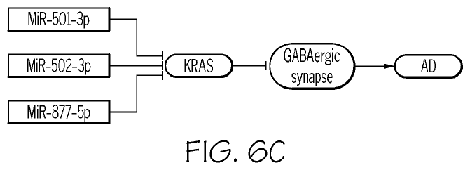

Lastly, Applicant studied the biological functions of synaptosome miRNAs which were deregulated in AD vs UC. The miR-500 family (miR-501-3p, miR-500a-3p) and miR-877-5p were identified to be significantly (P<0.05) involved in several biological process and disorders. MiRNA and disease interaction analysis showed a significant connection of miR-501-3p in GABAergic synapse function and other brain functions (FIG. 6A). The miRNAs target predication analysis showed more than 20 genes that are targeted by these miRNAs (FIG. 6B). The KRAS gene was identified as one of the potential common targets of both miR-501-3p and miR-877-5p. Further, gene ontology enrichment analysis of miR-502-3p showed that it is involved in several biological processes, cellular components and molecular functions. The most significant involvement were responses to external stimuli (P=0.009) and nervous system development (P=0.044). The most significant cellular component was GABAergic synapse (P=0.028), and molecular function was calmodulin binding (P=0.020).

Overall IP and gene ontology enrichment analysis showed that synaptosome miRNAs are altered in several neurological disorders and participates in numerous cellular and molecular pathways related to brain function.

### Example 1.23. Discussion

Synapses are essential for healthy brain activity. Synapse dysfunction is considered as the first stage of developing clinical symptoms of dementia. A balanced regulation of synaptic activity is critical for healthy synapse function. Synapse components can be extracted from postmortem brains in an intact form referred to as ‘synaptosome or synapto-neurosomes.’ Synaptosomes are the best neural cell component to study the synapse dysfunction in multiple neurodegenerative diseases, particularly in AD, where the synaptosome structure and functions are altered due to Aβ and p-tau accumulations. Although, significant research has been done on synaptosomal function and dysfunction, knowledge is limited on physiological connections and pathological changes in AD, particularly sequence of events that occur at synapse and the regulation of miRNAs synaptosomes and how synaptosomal miRNAs different from cytosolic miRNAs.

Using global synaptosomal and cytosolic microRNA analysis, in-silico analysis, transmission electron microscopy of healthy and AD postmortem brains and brain tissues from APP and Tau transgenic mice, in the current study Applicant investigated a comprehensive synaptic and cytosolic miRNAs. Applicant also determined the possible molecular function of synaptic miRNAs in AD and brain aging.

miRNAs are present in different cell organelles and cellular components such as the nucleus, mitochondria, Golgi bodies, exosomes and apoptotic bodies. These differentially existed miRNAs can modulate the levels of localized proteins. Therefore, Applicant hypothesized that there are also synapse centered miRNAs alter in AD. Applicant also hypothesized that miRNAs in synaptosomes and cytosols are differently expressed and localized in healthy (unaffected controls) and AD states. Therefore, for the first time, Applicant's study distinguished the cytosolic and synaptosomal miRNAs and their alterations in healthy and AD states.

Applicant examined the cytosolic and synaptosomal miRNAs changes in both healthy and disease states. In primary screenings, some individual synaptosomal and cytosolic miRNAs were identified as those which were expressed in both healthy and disease states but with varying expression levels, in terms of fold change (≤−2 and ≥2). Applicant noted that fold change of similar synaptosomes miRNAs varied by >100-folds in AD relative to healthy state. Most of these synaptosomal miRNAs are studied in human diseases, but very limited information is available on the cytosolic miRNAs.

As shown by pie chart analysis, more than 99% of miRNA population did not show significant changes in synaptosome and cytosol. Only a small fraction (<1%) of miRNA pool showed the significant changes among synaptosome and cytosol populations. MiR-107 appeared in healthy synaptosome but not in AD, while miR-151a-5p was significantly expressed in AD synaptosome but not in healthy state. These findings confirmed that most of the miRNA populations are uniformly distributed in the neuron with an exception of some localized synapse miRNAs. These synaptosome miRNAs are either synthesized locally at the synapse or may be transported from the soma to synapse. As per Applicant's initial analysis, miRNA biogenesis machinery may be present at synapse, and that miRNAs processing may occur at the synapse.

Validation analysis on the postmortem brains and brain tissues from AD mouse models amplified only limited numbers of miRNAs compared to primary Affymetrix screening. Applicant's extensive and careful validation analysis of postmortem brains revealed several potential miRNAs that showed similar expression trends specified as synaptosome or cytosolic miRNAs in both healthy and AD states. Further, extended validation analysis of APP Tg and Tau Tg mice shortlisted quite a few specific miRNAs. Overall, human samples and mouse data analysis revealed ten potential miRNAs designated as synaptosome miRNAs-miR-17-5p, let-7d-5p, miR-185-5p, miR-103-3p, miR-138-5p, miR-877-5p, miR-24-3p, miR-502-3p, miR-140-3p and miR-124-3p.

Most of these miRNAs, including miR-501-3p, miR-185-5p, miR-103-3p, miR-138-5p, miR-140-3p and miR-124-3p are actively involved in several neural functions. The other miRNAs-let-7d-5p, miR-877-5p, and miR-24-3p are not explored in AD thus far.

Data was obtained in the case of cytosolic miRNAs in AD vs healthy controls. The initial screening showed reduced expression of all cytosolic miRNAs in AD cytosol. Without being bound by theory, such observations could be because of higher AR and p-tau concentration in the cytoplasm compared to the synapse and high toxicities may be responsible for altered expression of miRNAs.

Applicant's validation analysis using postmortem brains, WT mice, APP Tg and Tau Tg mice strongly unveiled miR-638 and miR-3656 as potential cytosolic miRNAs. Both miRNAs are unique in AD and need further investigation on cytosolic basis of AD progression.

The top synaptosomal miRNAs are miR-500a-3p, miR-501-3p, miR-502-3p and miR-877-5p. In addition, most down regulated miRNA was miR-4499 as shown by primary screening. MiR-500 cluster miRNAs were amplified in all validation settings. However, Applicant did not see any significant expression of miR-4499 in the validation phase. The Gene Ontology Enrichment and IP analysis for the miR-500 cluster showed that miR-500 family involved in key biological process, cellular function and molecular function.

The most significant biological process is response to external stimulus and most significant cellular component is GABAergic synapse. GABAergic synapse is a crucial inhibitory synapse that are dysfunctional in AD. Applicant's results also confirmed the reduced levels of GABRA1 in AD synaptosomes. Further, in silico analysis showed that miR-502-3p could modulate the function of GABAergic synapse. Both Gene Ontology and IP analysis confirmed the strong links of miR-501-3p and miR-502-3p in GABAergic synapse pathways. Such activities could be mediated via modulation of KRAS gene by these miRNAs (FIG. 6C). Further, miR-501-3p, miR-502-3p and miR-877-5p expression was significantly increased with Braak stages of AD postmortem brains again confirmed the strong connection of these miRNAs with AD.

A recent study found that knockout of miR-500 rescued the GABAergic synapses in the spinal dorsal horn neurons in rats with neuropathic pain. Another study revealed miR-501-3p increased locally in dendrites after NMDAR activation and up-regulation of miR-501-3p is required for NMDAR dependent inhibition of excitatory synapse GluA1 expression. Since there is no report on the miR-500 family, synapse function in AD and miR-501-3p and miR-502-3p expression is inversely correlated with excitatory and inhibitory synapses. It is important to study the roles of miR-500, miR-501-3p and miR-502-3p in the regulation of excitatory and inhibitory synapse function in relation to AD.

In summary, Applicant's results in this Example identified synaptosome miRNAs that are deregulated in AD. Applicant's comprehensive analysis identified the two most promising synaptosomal miRNAs-miR-501-3p and miR-502-3p that could modulate the function of excitatory and inhibitory synapses in AD.

### Example 2. Impact of miR-502-3p on Cell Viability and Gene Expression

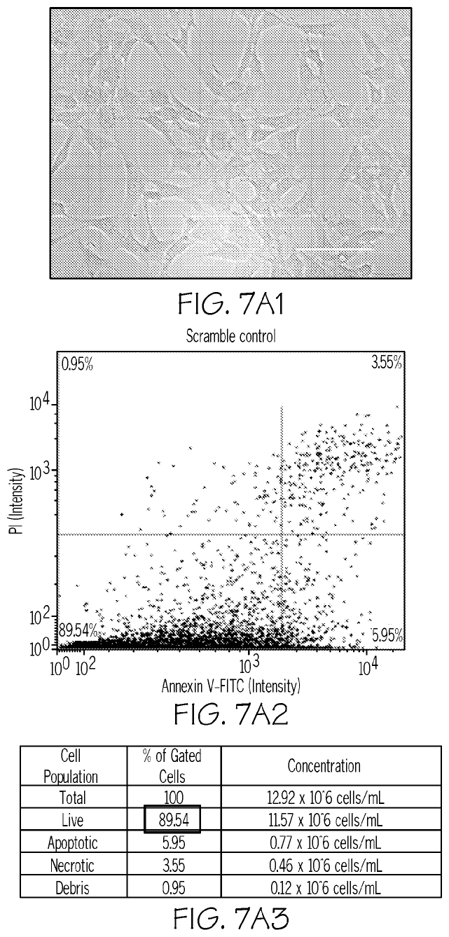

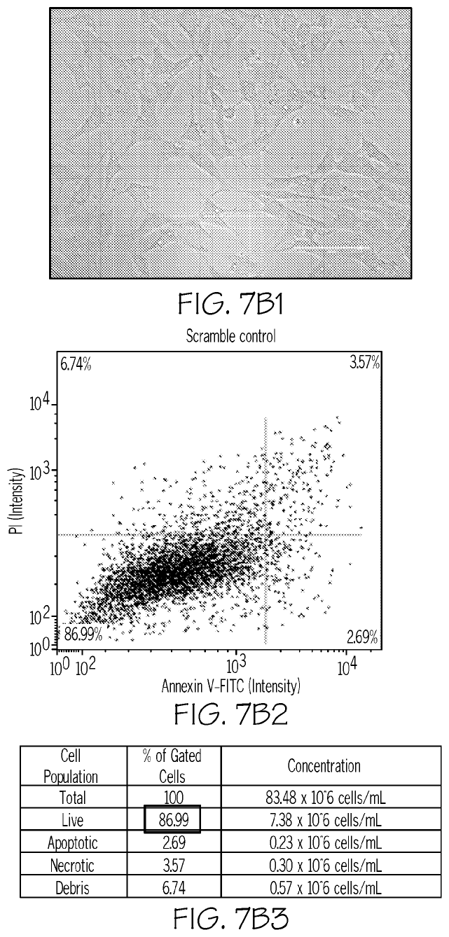

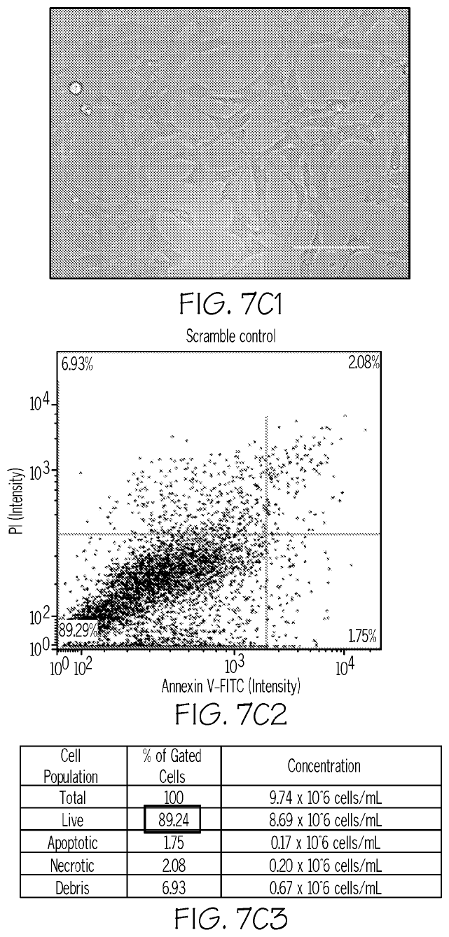

In this Example, Applicant provides experimental results related to the effect of miR-502-3p on cell viability and gene expression. FIGS. 7A1-7C3 summarize experimental results related to the impact of miR-502-3p on cell viability and cell survival of mouse primary hippocampal neurons. In particular, the results demonstrate that miR-502-3p overexpression reduces the cells viability and miR-502-3p inhibition improved the cell survival. Therefore, miR-502-3p has protective roles in cell viability and cell survival.

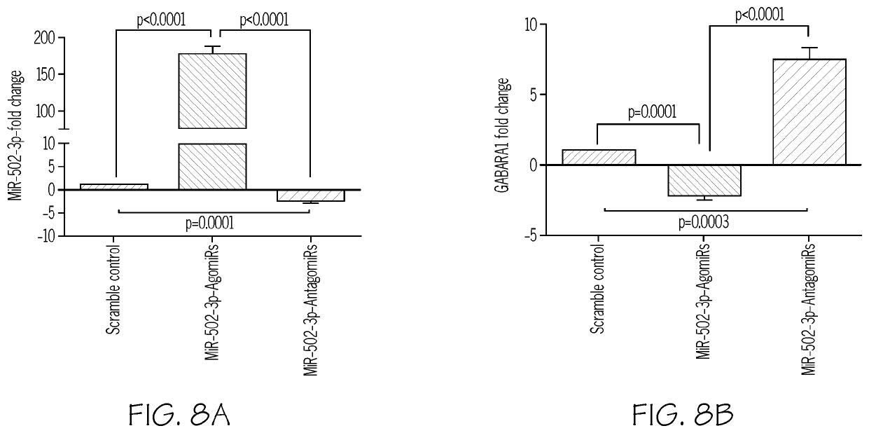

FIGS. 8A-8B summarize experimental results related to the impact of miR-502-3p on GABAergic receptor 1 alpha (GABARA1) expression. In particular, the results summarize qRT-PCR analysis of miR-502-3p and GABARA1 genes in HT22 cells. The results indicate that miR-502-3p overexpression reduces the expression of GABARA1 gene and miR-502-3p inhibition increases the expression of GABARA1 gene. Therefore, inhibition of miR-502-3p increased the GABAergic synapse function.

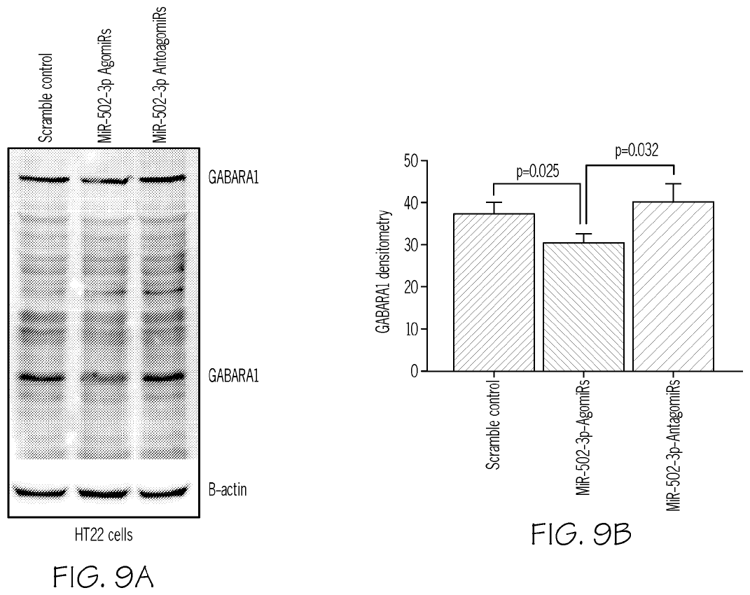

FIGS. 9A-9B summarize experimental results related to the impact of miR-502-3p on GABAergic receptor 1 alpha (GABARA1) protein levels. In particular, the results summarize immunoblot analyses of GABARA1 proteins in HT22 cells. The results indicate that miR-502-3p overexpression reduces the levels of GABARA1 protein and miR-502-3p inhibition increases the levels of GABARA1 protein.

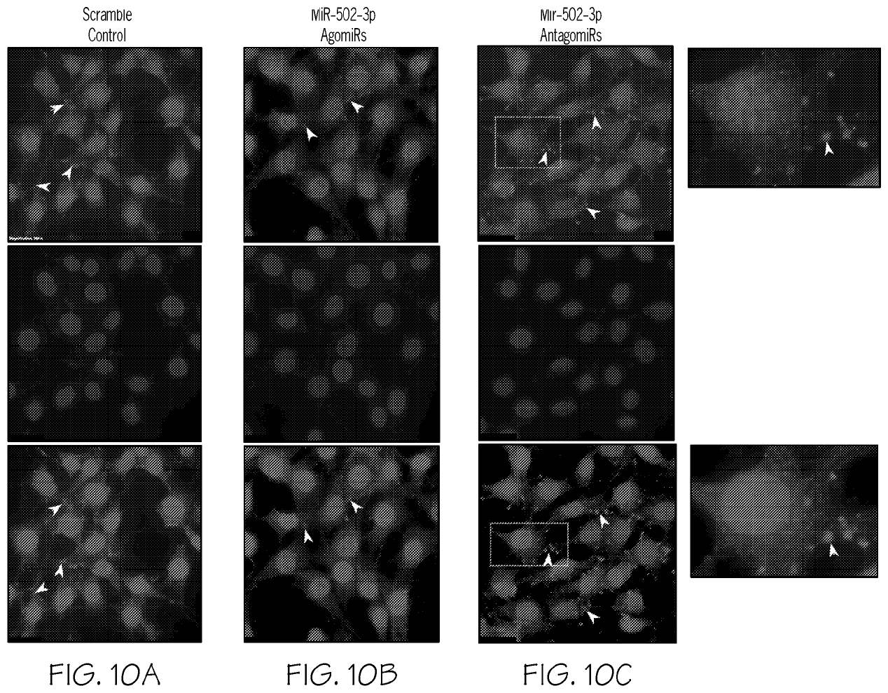

FIGS. 10A-10C summarize experimental results related to the impact of miR-502-3p on GABAergic receptor 1 alpha (GABARA1) protein levels. In particular, the results summarize immunostaining analyses of GABARA1 proteins in HT22 cells. The arrows show the GABARA1 proteins in the cytoplasm, cell junctions and synapses. The results indicate that miR-502-3p overexpression reduces the levels of GABARA1 protein and miR-502-3p inhibition increases the levels of GABARA1 protein in cells cytoplasm and cell junctions and synapses. Therefore, miR-502-3p inhibitors suppress the miR-502-3p at cell junction and increased the GABARA1 levels.

FIGS. 11A-11D summarize experimental results related to the impact of miR-502-3p on GABAergic receptor 1 alpha (GABARA1) protein levels. In particular, the results summarize confocal microscopic analyses of GABARA1 proteins in HT22 cells. The results indicate that miR-502-3p overexpression reduces the levels of GABARA1 protein at cell junctions and miR-502-3p inhibition increases the levels of GABARA1 protein and cell junctions and synapses Therefore, miR-502-3p inhibitors suppress the miR-502-3p at cell junction and increased the GABARA1 levels.

### Example 3. Modulation of GABA Function by miR-502-3p and its Inhibitor

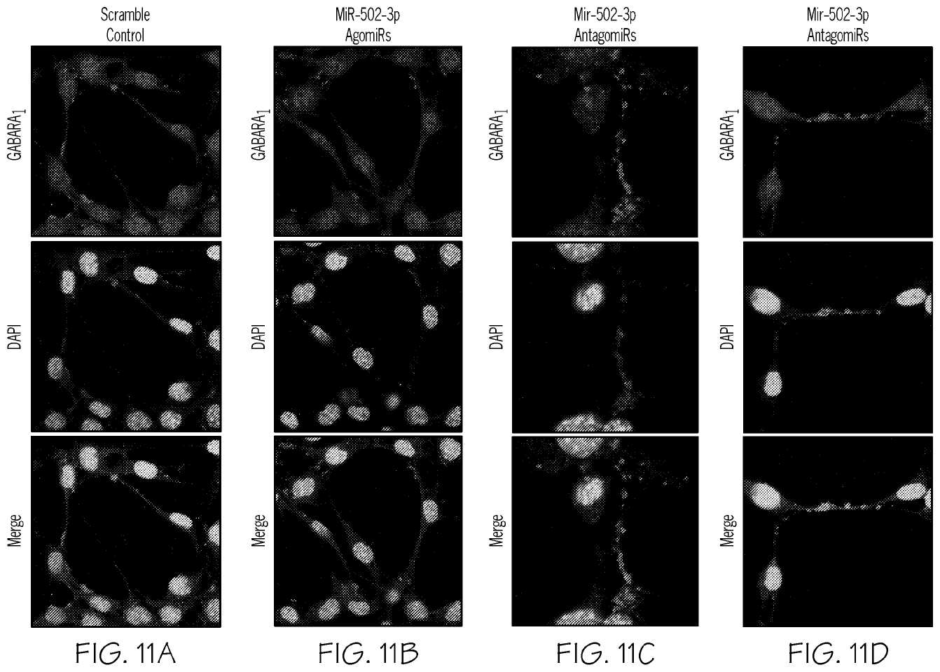

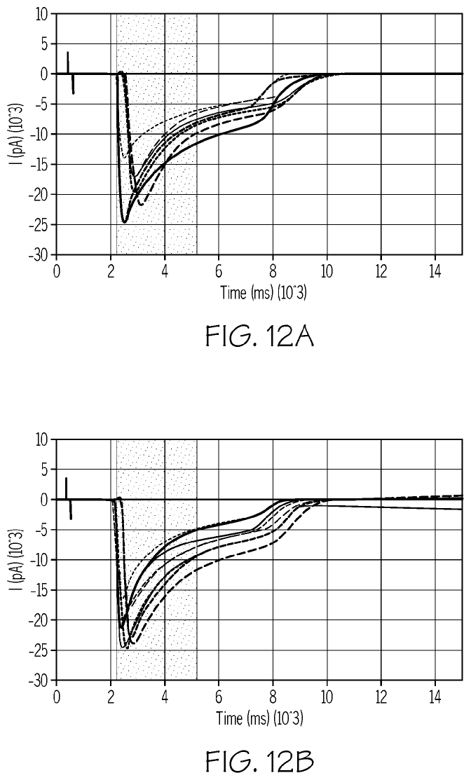

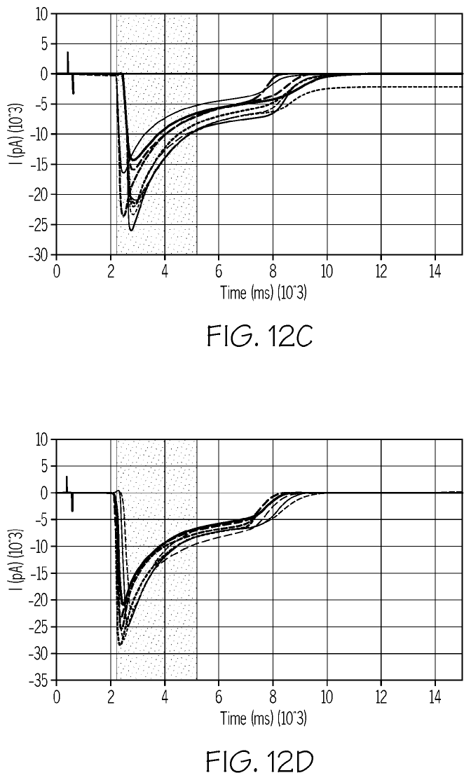

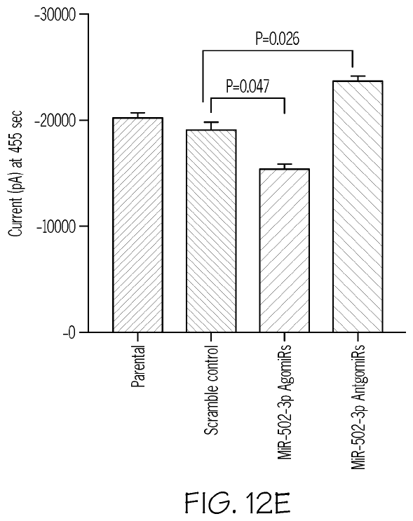

This Example provides data related to the modulation of GABA function by miR-502-3p and its inhibitor. FIGS. 12A-12E provide electrophysiology-patch clamp analyses. In the experiments, in-cells were transfected with miR-502-3p agomiRs (mimics; SEQ ID NO: 1) and antagomiRs (inhibitors; SEQ ID NO: 31). The patch clamp analysis of miR-502-3p agomiRs treated cells showed the reduced GABA current (FIG. 12C) while miR-502-3p antagomiRs treated cells showed increased GABA current (FIG. 12D). These observations confirm that miR-502-3p could modulate GABA function. FIG. 12A shows a parental control and FIG. 12B shows a scramble control.

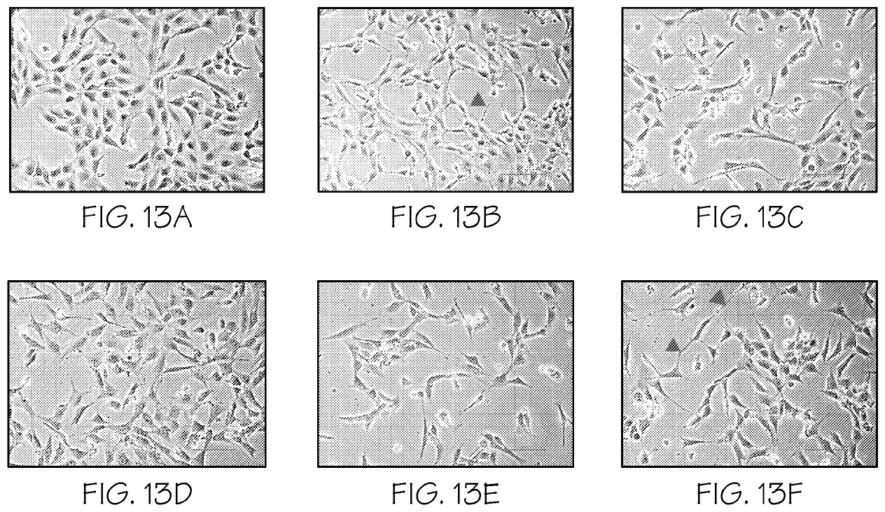

FIGS. 13A-13F show light microscopy images of mouse hippocampal neurons (HT22 cells). Shown are cell morphologies of HT22 cells transfected with GABRA1 expression vector (FIG. 13B), miR-502-3p overexpression vector (FIG. 13C) and miR-502-3p sponge suppression plasmids (FIG. 13)). GABARA1 increases the cell viability and dendrites formation while miR-502-3p overexpression reduced the cell viability and dendrites formation. MiR-502-3p suppression increased cell viability and dendritic formations. FIG. 13A shows a scramble control. FIG. 13E shows cells co-transfected with miR-502-3p and GABRA1 expression vectors. FIG. 13F shows cells co-transfected with GABRA1 expression vector and miR-502-3p sponge suppression plasmids.

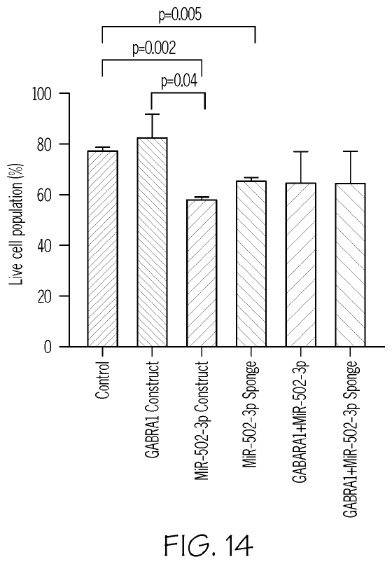

FIG. 14 summarizes cell viability analyses of HT22 cells transfected with GABRA1 expression vector, miR-502-3p overexpression vector, and miR-502-3p sponge suppression plasmids. GABRA1 overexpression increased the cell viability while miR-502-3p overexpression reduced the cell viability. MiR-502-3p suppression further significantly increased the cell viability.

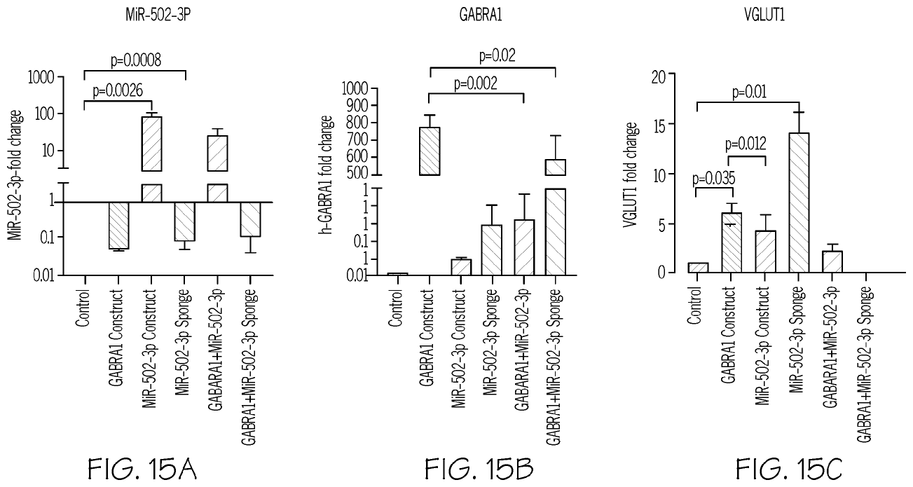

FIGS. 15A-15C summarize QRT-PCR analysis of miR-502-3p, GABRA1 and VGLUT1 in cells transfected with GABRA1 expression vector, miR-502-3p overexpression vector and miR-502-3p sponge suppression plasmids. MiR-502-3p overexpression reduced the GABRA1 and VGLUT1 levels, while suppression of miR-502-3p via miR-502-3p sponge increased the expression levels of both GABRA1 and VGLUT1.

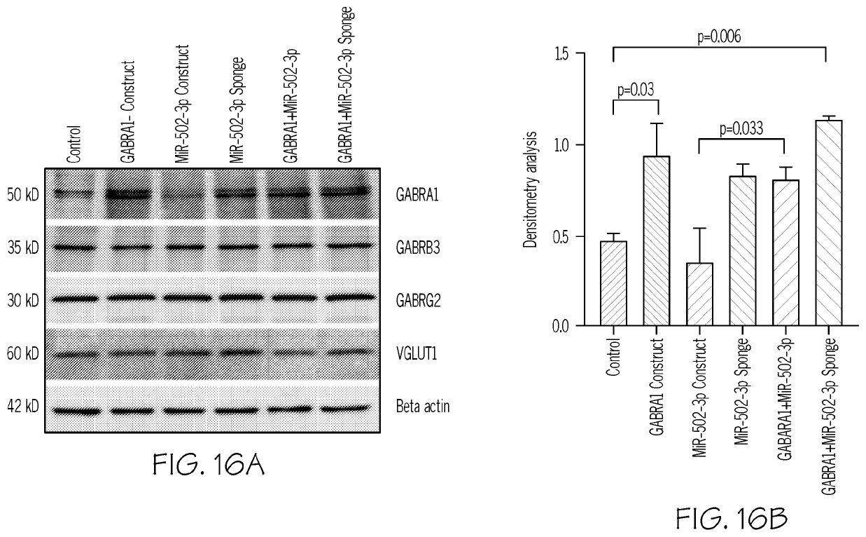

FIGS. 16A-16B summarize immunoblot (FIG. 16A) and quantitative (FIG. 16B) analyses of GABRA1 in HT22 cells transfected with GABRA1 overexpression vector, miR-502-3p overexpression vector and miR-502-3p sponge suppression plasmids. MiR-502-3p overexpression reduced the GABRA1 protein levels, while suppression of miR-502-3p via miR-502-3p sponge, increased the levels of GABRA1 protein. Therefore, miR-502-3p suppression could be a therapeutic option to restore the GABAergic synapse dysfunction in Alzheimer's disease.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. A method of treating or preventing a neurological disorder in a subject, wherein the method comprises:
administering to the subject at least one microRNA (miRNA), at least one inhibitor of the at least one miRNA, or combinations thereof,
wherein the at least one miRNA is selected from the group consisting of miR-501-3p (SEQ ID NO: 1), miR-502-3p (SEQ ID NO: 2), miR-877-5p (SEQ ID NO: 3), miR-500a-3p (SEQ ID NO: 4), miR-664b-3p (SEQ ID NO: 5), miR-4508 (SEQ ID NO: 6), miR-1237-5p (SEQ ID NO: 7), miR-5001-5p (SEQ ID NO: 8), miR-4497 (SEQ ID NO: 9), miR-103-3p (SEQ ID NO: 10), miR-103a-3p (SEQ ID NO: 11), miR-124-3p (SEQ ID NO: 12), miR-24-3p (SEQ ID NO: 13), let-7a-5p (SEQ ID NO: 14), miR-185-5p (SEQ ID NO: 15), miR-320b (SEQ ID NO: 16), let-7d-5p (SEQ ID NO: 17), miR-140-3p (SEQ ID NO: 18), miR-17-5p (SEQ ID NO: 19), miR-151a-5p (SEQ ID NO: 20), miR-3196 (SEQ ID NO: 21), miR-6511b-5p (SEQ ID NO: 22), miR-4508 (SEQ ID NO: 23), miR-1237-3p (SEQ ID NO: 24), miR-5001-5p (SEQ ID NO: 25), miR-4492 (SEQ ID NO: 26), miR-4499 (SEQ ID NO: 27), miR-4497 (SEQ ID NO: 28), miR-107 (SEQ ID NO: 29), miR-138-5p (SEQ ID NO: 30), derivatives thereof, or combinations thereof.

2. The method of claim 1, wherein the administering comprises administering at least one miRNA.

3. The method of claim 2, wherein the at least one miRNA is selected from the group consisting of miR-3196 (SEQ ID NO: 21), miR-6511b-5p (SEQ ID NO: 22), miR-4508 (SEQ ID NO: 23), miR-1237-5p (SEQ ID NO: 7), miR-5001-5p (SEQ ID NO: 8), miR-4492 (SEQ ID NO: 26), miR-4499 (SEQ ID NO: 27), miR-4497 (SEQ ID NO: 28), miR 107 (SEQ ID NO: 29), derivatives thereof, or combinations thereof.

4. The method of claim 1, wherein the administering comprises administering at least one inhibitor of the at least one miRNA.

5. The method of claim 4, wherein the at least one inhibitor is selected from the group consisting of a miR-501-3p inhibitor, a miR-502-3p inhibitor, a miR-877-5p inhibitor, a miR-500a-3p inhibitor, a miR-664b-3p inhibitor, a miR-4508 inhibitor, a miR-1237-5p inhibitor, a miR-5001-5p inhibitor, a miR-4497 inhibitor, a miR-103-3p inhibitor, a miR-103a-3p inhibitor, a miR-124-3p inhibitor, a miR-24-3p inhibitor, a let-7a-5p inhibitor, a miR-185-5p inhibitor, a miR-320b inhibitor, a let-7d-5p inhibitor, a miR-140-3p inhibitor, a miR-17-5p inhibitor, a miR-151a-5p inhibitor, or combinations thereof.

6. The method of claim 4, wherein the at least one inhibitor comprises a miR-501-3p inhibitor.

7. The method of claim 4, wherein the at least one inhibitor comprises a miR-502-3p inhibitor.

8. The method of claim 4, wherein the at least one inhibitor comprises a miR-877-5p inhibitor.

9. The method of claim 4, wherein the at least one inhibitor comprises a miR-500a-3p inhibitor.

10. The method of claim 1, wherein the neurological disorder is selected from the group consisting of Alzheimer's Disease (AD), Huntington's Disease, Parkinson's Disease, Amyotrophic Lateral Sclerosis (ALS), Multiple Sclerosis (MS), dementia, mild cognitive impairment (MCI), Schizophrenia, or combinations thereof.

11. The method of claim 1, wherein the neurological disorder is Alzheimer's Disease (AD).

12. The method of claim 1, wherein the administering occurs by a method selected from the group consisting of intravenous administration, subcutaneous administration, transdermal administration, topical administration, intraarterial administration, intrathecal administration, intracranial administration, intraperitoneal administration, intraspinal administration, intranasal administration, intraocular administration, oral administration, intratumor administration, or combinations thereof.

13. The method of claim 1, wherein the administering occurs by intracranial administration.

14. The method of claim 1, wherein the method prevents the neurological disorder.

15. The method of claim 1, wherein the method treats the neurological disorder.

16. The method of claim 1, wherein the method treats and prevents the neurological disorder.

17. The method of claim 1, wherein the subject is a human being.

18. The method of claim 1, wherein the subject is suffering from the neurological disorder.

19. The method of claim 1, wherein the subject is vulnerable to the neurological disorder.

20. The method of claim 1, wherein the subject exhibits symptoms of the neurological disorder.

21. A composition comprising at least one microRNA (miRNA), at least one inhibitor of the at least one miRNA, or combinations thereof, wherein the at least one miRNA is selected from the group consisting of miR-501-3p (SEQ ID NO: 1), miR-502-3p (SEQ ID NO: 2), miR-877-5p (SEQ ID NO: 3), miR-500a-3p (SEQ ID NO: 4), miR-664b-3p (SEQ ID NO: 5), miR-4508 (SEQ ID NO: 6), miR-1237-5p (SEQ ID NO: 7), miR-5001-5p (SEQ ID NO: 8), miR-4497 (SEQ ID NO: 9), miR-103-3p (SEQ ID NO: 10), miR-103a-3p (SEQ ID NO: 11), miR-124-3p (SEQ ID NO: 12), miR-24-3p (SEQ ID NO: 13), let-7a-5p (SEQ ID NO: 14), miR-185-5p (SEQ ID NO: 15), miR-320b (SEQ ID NO: 16), let-7d-5p (SEQ ID NO: 17), miR-140-3p (SEQ ID NO: 18), miR-17-5p (SEQ ID NO: 19), miR-151a-5p (SEQ ID NO: 20), miR-3196 (SEQ ID NO: 21), miR-6511b-5p (SEQ ID NO: 22), miR-4508 (SEQ ID NO: 23), miR-1237-3p (SEQ ID NO: 24), miR-5001-5p (SEQ ID NO: 25), miR-4492 (SEQ ID NO: 26), miR-4499 (SEQ ID NO: 27), miR-4497 (SEQ ID NO: 28), miR-107 (SEQ ID NO: 29), miR-138-5p (SEQ ID NO: 30), derivatives thereof, or combinations thereof.

22. The composition of claim 21, wherein the composition is suitable for use in treating or preventing a neurological disorder in a subject.

23. The composition of claim 21, wherein the composition comprises at least one miRNA.

24. The composition of claim 23, wherein the at least one miRNA is selected from the group consisting of miR-3196 (SEQ ID NO: 21), miR-6511b-5p (SEQ ID NO: 22), miR-4508 (SEQ ID NO: 23), miR-1237-5p (SEQ ID NO: 7), miR-5001-5p (SEQ ID NO: 8), miR-4492 (SEQ ID NO: 26), miR-4499 (SEQ ID NO: 27), miR-4497 (SEQ ID NO: 28), miR 107 (SEQ ID NO: 29), derivatives thereof, or combinations thereof.

25. The composition of claim 21, wherein the composition comprises at least one inhibitor of the at least one miRNA.

26. The composition of claim 25, wherein the at least one inhibitor is selected from the group consisting of a miR-501-3p inhibitor, a miR-502-3p inhibitor, a miR-877-5p inhibitor, a miR-500a-3p inhibitor, a miR-664b-3p inhibitor, a miR-4508 inhibitor, a miR-1237-5p inhibitor, a miR-5001-5p inhibitor, a miR-4497 inhibitor, a miR-103-3p inhibitor, a miR-103a-3p inhibitor, a miR-124-3p inhibitor, a miR-24-3p inhibitor, a let-7a-5p inhibitor, a miR-185-5p inhibitor, a miR-320b inhibitor, a let-7d-5p inhibitor, a miR-140-3p inhibitor, a miR-17-5p inhibitor, a miR-151a-5p inhibitor, or combinations thereof.

27. The composition of claim 25, wherein the at least one inhibitor comprises a miR-501-3p inhibitor.

28. The composition of claim 25, wherein the at least one inhibitor comprises a miR-502-3p inhibitor.

29. The composition of claim 25, wherein the at least one inhibitor comprises a miR-877-5p inhibitor.

30. The composition of claim 25, wherein the at least one inhibitor comprises a miR-500a-3p inhibitor.
